(19) United States
(12) Patent Application Publication
(10) Pub. No.: US 2010/0158822 A1
(43) Pub. Date: Jun. 24, 2010

(54) PEPTIDES THAT BIND TO SILICA-COATED PARTICLES

(75) Inventors: STEPHEN R. FAHNESTOCK, WILMIMGTON, DE (US); KARI A. FOSSER, WILMINTON, DE (US); ANJU PARTHASARATHY, GLENMOORE, PA (US); HONG WANG, KENNETT SQUARE, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E .I. DU PONT DE NEMOURS AND COMPANY, WILMINGTON, DE (US)

(21) Appl. No.: 12/632,829

(22) Filed: Dec. 8, 2009

Related U.S. Application Data
(60) Provisional application No. 61/138,631, filed on Dec. 18, 2008.

(51) Int. Cl. A61K 8/64; C07K 7/08; A61K 38/10; A61Q 11/00

(52) U.S. Cl. 424/49; 530/326; 514/13; 514/14

(57) ABSTRACT

Peptides having strong affinity for silica as well as surfaces comprising silica are described. The silica-binding peptides may be used to construct peptide based-reagents suitable for delivery of a silica-coated particulate benefit agent to a surface, such as body surface, for personal care and cosmetic applications. Peptide-based reagents formed by coupling at least one of the silica-binding peptides to at least one body surface-binding peptide, either directly or through a spacer, are described. The peptide-based reagents may be used in conjunction with at least one silica-coated colorant to color body surfaces.

### CROSS-REFERENCE TO A RELATED APPLICATION

This application claims the benefit of U.S. Provisional Patent Application No. 61/138,631 filed Dec. 18, 2008, incorporated herein by reference.

### FIELD OF THE INVENTION

The invention relates to the field of personal care products. More specifically, the invention relates to peptide-based reagents comprising at least one body surface-binding peptide and at least one of the present silica-binding peptides as well as personal care compositions comprising such materials. A method of delivering a silica-coated particulate benefit agent a body surface using one of the present peptide-based body surface reagents is also provided.

### BACKGROUND OF THE INVENTION

Proteinaceous materials coupled to one or more cosmetic care benefit agents have been reported in the art. Lang et al. in U.S. Pat. No. 5,192,332 describe temporary coloring compositions that contain an animal or vegetable protein, or hydrolysate thereof, which contain residues of dye molecules (e.g., benefit agents) grafted onto the protein chain. In the Lang et al. compositions, the protein serves as a conditioning agent and does not provided targeted delivery or enhanced durability for coupling the benefit agent to the target surface.

Proteinaceous materials having strong affinity for a body surface have been used for targeted delivery of one or more personal care benefit agents. However, many of these materials used for targeted delivery are comprised or derived from immunoglobulins or immunoglobulin fragments (antibodies, antibody fragments, F_{ab}, single-chain variable fragments (scFv), and Camelidae V_{HH}) having affinity for the target surface. For example, Horikoshi et al. in JP 08104614 and Igarashi et al. in U.S. Pat. No. 5,597,386 describe hair coloring agents that consist of an anti-keratin antibody covalently attached to a dye or pigment. The antibody binds to the hair, thereby enhancing the binding of the hair coloring agent to the hair. Similarly, Kizawa et al. in JP 09003100 describe an antibody that recognizes the surface layer of hair and its use to treat hair. A hair coloring agent consisting of that anti-hair antibody coupled to colored latex particles is also described. The use of antibodies to enhance the binding of dyes to the hair is effective in increasing the durability of the hair coloring material. However, the use of antibodies may not be possible in personal care products as they are difficult and expensive to produce.

Terada et al. in JP 2002363026 describe the use of conjugates consisting of single-chain antibodies, preferably anti-keratin, coupled to dyes, ligands, and cosmetic agents for skin and hair care compositions. Although single-chain antibodies may be prepared using genetic engineering techniques, these molecules are expensive to prepare and may not be suitable for use in commercial personal care products due to their conserved structure (i.e., immunoglobulin folds) and large size.

Non-immunoglobulin derived scaffold proteins have also been developed for targeted delivery of benefit agents to a target surface, such as delivery of cosmetic agents to keratin-containing materials (See Binz, H. et al. (2005) Nature Biotechnology 23, 1257-1268 for a review of various proteins used in scaffold-assisted binding). Findlay in WO 00/048558 describes the use of calycin-like scaffold proteins, such as β-lactoglobulin, which contain a binding domain for a cosmetic agent and another binding domain that binds to at least a part of the surface of a hair fiber or skin surface, for conditioners, dyes, and perfumes. Houtzager et al. in WO 03/050283 and US 2006/0140889 also describe affinity proteins having a defined core scaffold structure for controlled application of cosmetic substances. As with immunoglobulin-like proteins, these large scaffold protein are somewhat limited by the requirement to maintain the underlying conserved scaffold structure for effective binding and are expensive to produce.

Single chain peptide-based reagents lacking a scaffold support or immunoglobulin fold have been developed that can be used to couple benefit agents (such as colorants and conditioners) to a target surface. Examples of target surfaces include, but not are limited to body surfaces such as hair, skin, nail, and teeth (U.S. Pat. Nos. 7,220,405; 7,309,482; and 7,285,264; U.S. Patent Application Publication Nos. 2005-0226839; 2007-0196305; 2006-0199206; 2007-0065387; 2008-0107614; 2007-0110686; and 2006-0073111; and published PCT applications WO2008/054746; WO2004/048399, and WO2008/073368). However, the use of peptide-based reagents comprising one of the present silica-binding peptides for delivery of a silica-coated particle is not described.

European Patent EP1275728 B1 to Nomoto et al. describes peptides having high affinity for carbon black, copper phthalocyanine, titanium dioxide, and a silicon dioxide substrate. However, only two short peptides having affinity for silicon dioxide were reported. Nomoto et al. does not describe peptide-based body surface reagents or a method of delivering silica-coated particulate benefit agent to a body surface using the present peptide-based reagents.

Whaley et al. (Nature 405:626-627 (2000)) describes several peptides that bind to metals and metal oxides used in the semiconductor industry, such as gallium arsenide and silicon. Whaley et al. does not describe peptide-based body surface reagents for delivery of silica-coated particulate benefit agents for use in person care compositions.

Sarikaya et al. (Nat. Mater. (2003) 2:577-585) provides a comprehensive review of biomimetic nanostructures that can be achieved using peptides selected against various inorganic surfaces, including SiO₂, CaCO₃, and Fe₂O₃.

Naik et al. describes in WO2003/078451 (corresponding to U.S. Published Patent Application No. 2006-0035223) and in U.S. Published Patent Application No. 2006-0172282 several silica-binding peptides identified by phage display. However, Naik et al. does not describe use of silica-binding peptides in peptide-based reagents for use in personal care products.

In view of the above, a need exists to identify additional silica-binding peptides for use in peptide-based reagents for delivering silica-coated particulate benefit agents to body surfaces such as hair, skin, nails, and teeth. In a preferred embodiment, the silica-binding peptides are those capable of binding to the surface of a silica-coated particulate benefit agent under highly stringent conditions. In a preferred embodiment, the silica-binding peptides should be shampoo resistant.

Applicants have addressed the stated need by identifying peptide sequences that bind with high affinity to silica. One or more of the present silica-binding peptides can be coupled with one or more body surface-binding peptides to provide peptide-based body surface reagents that may be used in combination with a silica-coated particulate benefit agent in personal care compositions.

### SUMMARY OF THE INVENTION

Silica-binding peptides and peptide-based reagents comprising at least one of the present silica-binding peptides are provided. Peptide-based reagents comprising at least one body surface-binding peptide and at least one silica-binding peptide and may be used in conjunction with a silica-coated particulate benefit agent to couple the silica-coated benefit agent to a body surface.

In one embodiment, silica-binding peptide is provided having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, and 27.

In another embodiment, a peptide-based reagent is provided selected from the group consisting of:

a) a peptide-based reagent having the general structure:

[(BSBP)ₘ-(SiBP)ₙ]ₓ; and

b) a peptide-based reagent having the general structure:

[[(BSBP)ₘ-S_{q}]ₓ-[(SiBP)ₙ-Sᵣ]_{z}]_{y},;

wherein
i) BSBP is a body surface-binding peptide;
ii) SiBP is a silica-binding peptide having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, and 27;
iii) S is a spacer;
iv) m, n, x and z independently range from 1 to about 10;
v) y is from 1 to 5; and
vi) q an r are each independently 0 or 1, provided that both r and q may not be 0.

In another embodiment, a personal care composition is provided comprising at least one of the present peptide-based reagents and at least one silica-coated particulate benefit agent. In one aspect, the personal care composition is an oral care composition.

In another embodiment, a method for coupling a silica-coated particulate benefit agent to a body surface is provided comprising:
a) providing:
   i) at least one silica-coated particulate benefit agent; and
   ii) at least one peptide-based reagent; and
b) applying the at least one silica-coated particulate benefit agent of (a)(i) and the at least one peptide-based reagent of (a)(ii) to a body surface whereby the peptide-based reagent couples the silica-coated particulate benefit agent to the body surface.

In another embodiment, the above method further comprises the step of: c) applying at least one polymeric sealant to the body surface subsequent to step (b).

In one embodiment, the body surface is selected from the group consisting of hair, skin, nail, and tooth. In another embodiment, the body surface is tooth. In further embodiment, the body surface is tooth enamel or tooth pellicle.

In a preferred embodiment, the silica-coated particulate benefit agent is a silica-coated colorant.

### BRIEF DESCRIPTION OF THE BIOLOGICAL SEQUENCES

The invention can be more fully understood from the following detailed description and the accompanying sequence descriptions, which form part of this application.

The following sequences conform with 37 C.F.R. §§1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37C.F.R. §1.822.

SEQ ID NOS: 1-27 are the amino acid sequences of the present silica-binding peptides.

SEQ ID NOs: 28-34 and 298 are the amino acid sequences of various peptide-based reagents comprising one or more of the present silica-binding peptides and at least one body surface-binding peptide.

SEQ ID NOs: 35-161 are amino acid sequences of hair-binding peptides.

SEQ ID NOs: 157-209 are amino acid sequence of skin-binding peptides.

SEQ ID NOs: 210-211 are amino acid sequences of nail-binding peptides.

SEQ ID NOs: 212-251, 263-293, and 295-297 are amino acid sequences of tooth-binding peptides.

SEQ ID NO: 252 is the amino acid sequence of the Caspase 3 cleavage site.

SEQ ID NOs: 253-261 are the amino acid sequences of peptide spacers. SEQ ID NOs: 253-257 are examples of peptide linker sequences. SEQ ID NO: 258-261 are examples of peptide bridge sequences.

SEQ ID NO: 262 is the nucleic acid sequence of an oligonucleotide primer used to sequence phage DNA.

SEQ ID NO: 294 is the amino acid sequence of a polypeptide used as a control in Example 6.

### DETAILED DESCRIPTION OF THE INVENTION

Provided herein are peptide-based reagents comprising at least one body surface-binding peptide and at least one of the present silica-binding peptides. The peptide-based reagents may include one or more optional molecular spacers. The peptide-based reagents may be used in conjunction with at least one silica-coated particulate benefit agent to couple the silica-coated particulate benefit agent to a body surface. In one embodiment, the silica-coated particulate benefit agent comprises colorants (pigments, dyes and/or lakes), conditioning agents, antimicrobial agents, and pharmaceutically active benefit agents. In another embodiment, the silica-coated particulate benefit agent is a silica-coated pigment, such as silica-coated iron oxide or silica-coated titanium dioxide.

The following definitions are used herein and should be referred to for interpretation of the claims and the specification.

As used herein, the articles “a”, “an”, and “the” preceding an element or component of the invention are intended to be nonrestrictive regarding the number of instances (i.e., occurrences) of the element or component. Therefore “a”, “an” and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” refers to modifying the quantity of an ingredient or reactant of the invention or employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, the term “invention” or “present invention” is a non-limiting term and is not intended to refer to any single embodiment of the particular invention but encompasses all possible embodiments as described in the specification and the claims.

As used herein, the term “peptide” refers to two or more amino acids joined to each other by peptide bonds or modified peptide bonds.

As used herein, “TSBP” means target surface-binding peptide(s). The target surface-binding peptides described herein may refer to body surface-binding peptides and silica-binding peptides. The target-surface-binding peptides typically range in size from 7 to about 60 amino acids in length. Individual target surface-binding peptides will be referred to herein as a binding “fingers”. Linking together multiple “fingers” forms a binding domain or binding “hand” for the respective target surface. In one embodiment, each “finger” is a combinatorially-generated peptide isolated using biopanning, such as phage display or mRNA display

The term “body surface” refers to any surface of the human body that may serve as a substrate for the binding of a peptide-based reagent and a silica-coated particulate benefit agent. Typical body surfaces may include, but are not limited to hair, skin, nails, teeth, and tissues of the oral cavity, such as gums. In a preferred embodiment, the body surface is selected from the group consisting of hair, skin, nails, teeth, and tissues of the oral cavity, such as gums.

As used herein, “BSBP” may be used to refer to a body surface-binding peptide selected from the group consisting of hair-binding peptides, skin-binding peptides, nail-binding peptides, tooth-binding peptides, and peptides that have a specific affinity for oral cavity tissues, such as the gums. A body surface-binding peptide is a peptide that may range in size from about 7 amino acids to about 60 amino acids in length that binds with high affinity to at least one body surface. Each body-surface-binding peptide may be referred to herein as a binding “finger”. Multiple peptide “fingers” may be linked together to form a binding domain (a binding “hand”) having affinity for the respective body surface. In one embodiment, each “finger” may be a peptide isolated from a peptide library using biopanning.

As used herein, “SiBP” means silica-binding peptide. A silica-binding peptide is a target surface-binding peptide ranging in size from about 7 amino acids to about 60 amino acids in length that binds with high affinity to silica. In one embodiment, silica may be applied to a particulate benefit agent to provide a partial or complete silica coating on the particulate benefit agent (i.e., a “silica-coated particulate benefit agent”. In one embodiment, the silica-coated particulate benefit agent is a silica-coated pigment. Means to apply an effective amount of a silica coating to a particulate benefit agent, such as a pigment, are well-known in the art (see, for example, U.S. Pat. No. 2,885,366 to Iler).

As used herein, the terms “peptide-based reagent” and “peptide-based body surface reagent” will be used to refer to a single chain peptide comprising at least one portion having affinity for a body surface and at least one portion having affinity for a surface comprising an effective amount of silica. The peptide-based reagent may range in size from about 14 to about 600 amino acids in length and is not comprised of an immunoglobulin fold and does not require scaffold-assisted binding (i.e., the present peptide-based reagent is not comprised of a naturally-occurring scaffold protein used in scaffold-assisted binding; See Binz, H. et al. (2005) Nature Biotechnology 23, 1257-1268 for a review of various scaffold proteins used in scaffold-assisted binding). The peptide-based reagent is typically comprised of a first domain having affinity for a body surface and a second domain having affinity for a surface comprising silica. The peptide-based reagent may further comprise a “peptide bridge” separating the first domain from the second domain. The first and/or second domains may be comprised of a single peptide “finger” or may be comprised of a plurality of target surface-binding peptides, optionally separated with one or more peptide linkers.

As used herein, “S” means “spacer” or “linker”. Depending upon the location of the peptide spacer in the peptide-based reagent, the peptide spacer may also be referred to as a “peptide linker” or a “peptide bridge”. In one embodiment, the “spacer” may be a peptide linker. In another embodiment, the spacer may be a peptide bridge used to separate two or more binding domains.

As used herein, the term “pigment” refers to an insoluble, organic or inorganic colorant. It is a material that changes the color of light it reflects as the result of selective color absorption.

As used herein, the term “hair” as used herein refers to human hair, eyebrows, and eyelashes. As used herein, the term “hair-binding peptide” (HBP) refers to a peptide that binds with strong affinity to hair. Examples of hair-binding peptides are provided as SEQ ID NOs: 35-161. The hair-binding fingers may be linked together to form hair-binding domains (“hands”).

As used herein, the term “skin” as used herein refers to human skin, or substitutes for human skin, such as pig skin, VITRO-SKIN® (Innovative Measurement Solutions Inc., Milford, Conn.) and EPIDERM™ (MatTek Corporation, Ashland, Mass.). Skin, as used herein, will refer to a body surface generally comprising a layer of epithelial cells and may additionally comprise a layer of endothelial cells.

As used herein, the term “skin-binding peptide” (SBP) refers to a peptide that binds with strong affinity to skin. Examples of skin-binding peptides (“fingers”) have also been reported (U.S. patent application Ser. No. 11/069,858 to Buseman-Williams; WO 2004/000257 to Rothe et. al; U.S. patent application Ser. No. 11/696,380). Examples of skin-binding peptides are provided as SEQ ID NOs: 157-209. The skin-binding fingers may be linked together to form skin-binding domains (“hands”).

As used herein, the term “nails” as used herein refers to human fingernails and toenails. As used herein, the term “nail-binding peptide” (NBP) refers to a peptide that binds with strong affinity to nail. Examples of nail-binding peptides (“fingers”) are provided as SEQ ID NOs: 210-211. The nail-binding fingers may be linked together to form nail-binding domains (“hands”).

As used herein, the term “oral cavity surface-binding peptide” refers to a peptide that binds with strong affinity to surfaces such as teeth, gums, cheeks, tongue, or other surfaces in the oral cavity. In one embodiment, the oral cavity surface-binding peptide is a peptide that binds with strong affinity to a tooth surface.

As used herein, the term “tooth-binding peptide” (TBP) will refer to a peptide that binds with strong affinity to tooth enamel and/or tooth pellicle. Examples of biopanned tooth-binding peptides (“fingers”) having been disclosed in co-pending U.S. patent application Ser. No. 11/877,692 and U.S. Provisional Patent Application No. 61/164,476 and are provided herein as SEQ ID NOs: 212-251, 263-293, and 295-297. The tooth-binding fingers may be linked together to form tooth-binding domains (“hands”).

The term “tooth surface” will refer to a surface comprised of tooth enamel (typically exposed after professional cleaning or polishing) or tooth pellicle (an acquired surface comprising salivary glycoproteins). Hydroxyapatite may be coated with salivary glycoproteins to mimic a natural tooth pellicle surface and may also be used for the identification of tooth-binding peptides (tooth enamel is predominantly comprised of hydroxyapatite).

As used herein, the terms “pellicle” and “tooth pellicle” will refer to the thin film (typically about 20 nm to about 200 μm in thickness) derived from salivary glycoproteins which forms over the surface of the tooth crown. Daily tooth brushing tends to only remove a portion of the pellicle surface while abrasive tooth cleaning and/or polishing will typically exposure more of the tooth enamel surface.

As used herein, the terms “enamel” and “tooth enamel” will refer to the highly mineralized tissue which forms the outer layer of the tooth. The enamel layer is composed primarily of crystalline calcium phosphate (i.e., hydroxyapatite) along with water and some organic material.

As used herein, the term “peptide linker” refers to a peptide ranging in size from 1 to 60 amino acids in length, preferably 3 to about 50 amino acids in length that is used to link together two target surface-binding peptides (“fingers”) to form a binding domain (“hand”). Examples of peptide linkers are provided as SEQ ID NOs: 253-257. In one embodiment, the peptide linker is the “TonB” linker provided as SEQ ID NO: 255.

As used herein, the term “peptide bridge” refers to a peptide ranging in size from 1 to 60 amino acids in length that is used to link together two binding domains (“hands”) or to link together a single binding “hand” directly to a benefit agent. Examples of peptide “bridges” are provided as SEQ ID NOs: 258-261.

As used herein, the terms “coupling” and “coupled” refer to any chemical association and includes both covalent and non-covalent interactions. In one embodiment, the silica-coated particulate benefit agent is coupled to a body surface by at least one of the present peptide-based reagents using non-covalent interactions.

The term “stringency” as it is applied to the selection of the target-surface-binding peptides, refers to the concentration of the eluting agent (usually detergent) used to elute peptides from the target surface. Higher concentrations of the eluting agent provide more stringent conditions. The

present silica-binding peptides were selected under highly stringent conditions (i.e., those resistant to washing conditions that included 0.5 wt % TWEEN® 20 and 30 wt % shampoo).

As used herein, the term “benefit agent” refers to colorants, whitening agents (e.g., bleaching agents, white pigments), conditioning agents, sunscreen agents, antimicrobial agents, nutrients (such as vitamins and essential oils), and pharmaceutically active benefit agents that are particulates and/or encapsulated on and/or in a particulate material (comprising an effective amount of silica). In one embodiment, the benefit agent is a particulate material and may be referred to herein as a “particulate benefit agent”. In another embodiment, the particulate benefit agent is a pigment coated with an effective amount of silica.

As used herein, the term “silica-coated particulate benefit agent” refers to a particulate benefit agent that is coated with silica or comprises a surface comprising silica in an amount capable of binding to one or the present silica-binding peptides. The particulate benefit agent may be partially or completely coated in silica so long as an effective amount of silica is present on the surface of the particle whereby the silica-coated particulate benefit agent binds with strong affinity to at least one of the present silica-binding peptides. Means to prepare silica-coated particles are well known in the art. For example, silica-coated particles can be made by the methods disclosed in U.S. Pat. No. 2,885,366 or U.S. Pat. No. 6,197,274.

As used herein, the term “effective amount of silica” or “effective amount of silicon dioxide” refers to an amount of silicon dioxide that provides a surface (comprising silica) capable of binding to one or more of the present silica-binding peptides, preferably with strong affinity. One of skill in the art can determine an effective amount by measuring an increase in binding of the silica-coated particulate benefit agent to a body surface when used in combination with one or more of the present peptide-based reagents comprising at least one silica-binding peptide, preferably at least one of the present silica-binding peptides.

As used herein, the term “MB₅₀” refers to the concentration of the binding peptide that gives a signal that is 50% of the maximum signal obtained in an ELISA-based binding assay (See Example 3). The MB₅₀ value provides an indication of the strength of the binding interaction. A lower MB₅₀ values correlate with a stronger binding affinity between the peptide and the respective substrate.

As used herein, the terms “binding affinity” and “affinity” refer to the strength of the interaction of a binding peptide (such as target surface-binding peptides, target surface-binding domains, and peptide reagents) with its respective substrate. The binding affinity may be reported in terms of the MB₅₀ value as determined in an ELISA-based binding assay or as a K_{D} (equilibrium dissociation constant) value, which may be deduced using surface plasmon resonance (SPR).

As used herein, the terms “strong affinity” and “high affinity” refer to a binding affinity, as measured as an MB₅₀ value of K_{D} value, of 10⁻⁴ M or less, preferably less than 10⁻⁶ M, more preferably less than 10⁻⁶ M, more preferably less than 10⁻⁷ M, even more preferably less than 10⁻⁸ M, and most preferably less than 10⁻⁹ M.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any naturally-occurring amino | Xaa | X |
| acid (or as defined herein) | | |

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

### Silica-Binding Peptides

Silica-binding peptides are peptide sequences that bind with strong affinity to a surface comprising silica (i.e., silicon dioxide; SiO₂). Particulate benefit agents comprising an effective amount of a silica, such as a silica coating, can be prepared that bind to one or more of the present silica-binding peptides. As such, peptides having strong affinity for a silica coating can be used to prepare peptide-based reagents capable of coupling a silica-coated particulate benefit agent to a body surface.

Peptides having an affinity for a target surface may be selected using combinatorial methods that are well-known in the art or may be empirically generated. The present silica-binding peptides typically have a binding affinity for silica, as measured by an MB₅₀ value or K_{D} value, of less than or equal to about 10⁻⁴ M, preferably less than or equal to about 10⁻⁶ M, more preferably less than or equal to about 10⁻⁶ M, more preferably less than or equal to about 10⁻⁷ M, even more preferably less than or equal to about 10⁻⁸ M, and even more preferably less than or equal to about 10⁻⁹ M. In one embodiment, the term “high affinity” or “strong affinity” will be used to describe silica-binding peptides having a binding affinity, as measured by MB₅₀, less than or equal to about 10⁻⁶M, preferably less than or equal to about 10⁻⁶ M, more preferably less than or equal to about 10⁻⁷M, and even more preferably less than or equal to about 10⁻⁸.

The present silica-binding peptides comprise an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, and 27.

### Identification of Additional Target Surface-Binding Peptides

Additional target surface-binding peptides (e.g., such as additional body surface-binding peptides that may be used in conjunction with the present silica-binding peptides to prepare peptide-based reagents) may be combinatorially-generated and may range in length from about 7 amino acids to about 60 amino acids, preferably 7 to 35 amino acids in length, and even more preferably from about 7 amino acids to about 20 amino acids in length. The target surface-binding peptides may be generated randomly (to form a peptide library) and then selected against the respective target surface, such as a body surface. Methods to identify and select peptides from peptide libraries may be accomplished by any number of techniques including, but not limited to, bacterial display (Kemp, D. J.; Proc. Natl. Acad. Sci. USA 78(7): 4520-4524 (1981); yeast display (Chien et al., Proc. Natl. Acad. Sci. USA 88(21): 9578-82 (1991)), combinatorial solid phase peptide synthesis (U.S. Pat. No. 5,449,754; U.S. Pat. No. 5,480,971; U.S. Pat. No. 5,585,275 and U.S. Pat. No. 5,639,603), phage display technology (U.S. Pat. No. 5,223,409; U.S. Pat. No. 5,403,484; U.S. Pat. No. 5,571,698; and U.S. Pat. No. 5,837,500), ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Techniques to generate such biological peptide libraries are described in Dani, M., J. of Receptor & Signal Transduction Res., 21(4):447-468 (2001). Additionally, phage display libraries are available commercially from companies such as New England BioLabs (Beverly, Mass.).

Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

More specifically, after a suitable library of peptides has been generated or purchased, the library is then contacted with an appropriate amount of the test substrate. The library of peptides is dissolved in a suitable solution for contacting the sample. The sample is typically suspended in solution or may be immobilized on a plate or bead. A preferred solution is a buffered aqueous saline solution containing a surfactant. A suitable solution is Tris-buffered saline (TBS) with 0.5% TWEEN® 20. The solution may additionally be agitated by any means in order to increase the mass transfer rate of the peptides to the target sample/surface, thereby shortening the time required to attain maximum binding.

Upon contact, a number of the randomly generated peptides will bind to the target surface to form a peptide-target surface complex, for example, a peptide-silica-coated pigment complex. Unbound peptide may be removed by washing. After all unbound material is removed, peptides having varying degrees of binding affinities for the test surface may be fractionated by selected washings in buffers having varying stringencies. Increasing the stringency of the buffer used increases the required strength of the bond between the peptide and target surface in the peptide-target surface complex.

A number of substances may be used to vary the stringency of the buffer solution in peptide selection including, but not limited to, acidic pH (1.5-3.0); basic pH (9-12.5); high salt concentrations such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P-40, Triton X-100, TWEEN® 20, wherein TWEEN® 20 is preferred. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred.

It will be appreciated that peptides having increasing binding affinities for target surface substrates may be eluted by repeating the selection process using buffers with increasing stringencies. The eluted peptides can be identified and sequenced by any means known in the art.

As many of the peptide-based reagents will be used in personal care products comprising significant amounts of surfactants/detergents (such as a shampoo), the stringency of the washing steps may be increased to select only those peptides having the highest binding affinity. In one embodiment, the washing conditions will include at least 1 wt % shampoo, preferably at least 5 wt %, even more preferably at least 10 wt %, even more preferably at least 20 wt %, and most preferably at least 30 wt % shampoo. In one embodiment, peptides that are resistant to washing conditions that includes a shampoo will be referred to herein as “shampoo resistant”. Particularly preferred peptides are those that are resistant to washing conditions that include at least 30 wt % shampoo (such as the present “shampoo-resistant silica-binding peptides”).

To identify peptide sequences that bind to one substrate but not to another, for example peptides that bind to another surface (i.e., a “non-target” surface; for example, another inorganic material or a body surface such as hair, skin, nail, teeth, etc.), a subtractive panning step may be added. Specifically, the library of combinatorially generated phage-peptides is first contacted with the non-target to remove phage-peptides that bind to it. Then, the non-binding phage-peptides are contacted with the desired substrate and the above process is followed. Alternatively, the library of combinatorially generated phage-peptides may be contacted with the non-target and the desired substrate simultaneously. Then, the phage-peptide-target surface complexes are separated from the phage-peptide-non-target complexes and the method described above is followed for the desired phage-peptide-target surface complexes.

In one embodiment, a modified phage display screening method for isolating peptides with a higher affinity for the target surface may be used. In the modified method, the phage-peptide-target surface complexes may be formed as described above. Then, these complexes are treated with an elution buffer. Any of the elution buffers described above may be used. Preferably, the elution buffer is an acidic solution. Then, the remaining, elution-resistant phage-peptide-target surface complexes are used to directly infect a bacterial host cell, such as E. coli ER2738. The infected host cells are grown in an appropriate growth medium, such as LB (Luria-Bertani) medium, and this culture is spread onto agar, containing a suitable growth medium, such as LB medium with IPTG (isopropyl β-D-thiogalactopyranoside) and S-GAL™. After growth, the plaques are picked for DNA isolation and are sequenced to identify the peptide sequences with a high binding affinity for the target surface of interest (for example, a silica coated particle).

In another embodiment, PCR may be used to identify the elution-resistant phage-peptides from the modified phage display screening method, described above, by directly carrying out PCR on the phage-peptide-target surface complexes using the appropriate primers, as described by Janssen et al. in U.S. Patent Application Publication No. 2003/0152976.

### Body Surfaces

A body surfaces is any surface on the human body that will serve as a substrate (i.e., binding target) for a body surface-binding peptide. The body surfaces are selected from the group consisting of hair, skin, nail, tooth, gums, and other tissues of the oral cavity. In many cases the body surfaces will be exposed to air, however in some instances, the oral cavity for example, the surfaces will be internal. Accordingly, body surfaces may include layers of both epithelial and well as endothelial cells.

Samples of body surfaces are available from a variety of sources. For example, human hair samples are available commercially, for example from International Hair Importers and Products (Bellerose, N.Y.), in different colors, such as brown, black, red, and blond, and in various types, such as African-American, Caucasian, and Asian. Additionally, the hair samples may be treated for example using hydrogen peroxide to obtain bleached hair. Human skin samples may be obtained from cadavers or in vitro human skin cultures. Additionally, pig skin, available from butcher shops and supermarkets, VITRO-SKIN®, available from IMS Inc. (Milford, Conn.), and EPIDERM™, available from MatTek Corp. (Ashland, Mass.), may be used as substitutes for human skin. Human fingernails and toenails may be obtained from volunteers. Extracted mammalian teeth, such as bovine and/or human teeth are commercially available. Extracted human teeth may also be obtained from dental offices. Bovine enamel may be sterilized and incubated in a human oral environment to form a pellicle layer on the enamel (Example 5). Additionally, hydroxyapatite, available in many forms, for example, from Berkeley Advanced Biomaterials, Inc. (San Leandro, Calif.), may be used once coated with salivary glycoproteins (to form an acquired pellicle) as a model for studying teeth-binding peptides (see U.S. Patent Application Publication No. 2008-0280810).

### Body Surface-Binding Peptides

Body surface-binding peptides as defined herein are peptide sequences that bind with strong affinity to a respective target body surface including, but not limited to hair, nail, skin, tooth, and tissues of the oral cavity (such as gums). In one embodiment, the body surface is a hair, skin, nail, or tooth surface. In one embodiment, the body surface-binding peptide are selected from the group consisting of hair-binding peptides, skin-binding peptides, nail-binding peptides, and tooth-binding peptides.

Phage display has been used to identify various body surface-binding peptides. For example, peptides having an affinity for a body surface have been described in U.S. Pat. Nos. 7,220,405 and 7,285,264; U.S. Patent Application Publications Nos. US 2005-0226839, US 2005-0249682, US 2006-0073111, US 2006-0199206, US 2007-0065387, US 2007-0067924, US 2007-0196305, US 2007-0110686, US 2008-0280810, and US 2008-0175798; and PCT Patent Application Publication No. WO2004048399.

Alternatively, hair-binding and skin-binding peptide sequences may be generated empirically by designing peptides that comprise positively charged amino acids, which can bind to hair and skin via electrostatic interaction, as described by Rothe et al. (WO 2004/000257). The empirically generated hair and skin-binding peptides have between 7 to about 60 amino acids, preferably about 4 amino acids to about 50 amino acids, more preferably from about 4 to about 25 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including solubility, viscosity and compatibility with other components in a formulated product and will therefore vary according to the needs of the application. In some cases the peptide may contain up to 60 mole % of amino acids not comprising histidine, lysine or arginine. Suitable empirically-generated hair-binding and skin peptides include, but are not limited to, SEQ ID NOs:157-161.

In one embodiment, the body surface-binding peptide is a hair-binding peptide selected from the group consisting of SEQ ID NOs: 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, and 161.

In one embodiment, the body surface-binding peptide is a skin-binding peptide selected from the group consisting of SEQ ID NOs: 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, and 209.

In one embodiment, the body surface-binding peptide is a nail-binding peptide selected from the group consisting of SEQ ID NOs: 210 and 211.

In one embodiment, the body surface-binding peptide is a tooth-binding peptide selected from the group consisting of SEQ ID NOs: 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 205, 251, 263, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 295, 296 and 297. In another embodiment, the tooth-binding peptide is selected from the group consisting of SEQ ID NOs: 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 295, 296 and 297.

### Peptide-Based Body Surface Reagents

The peptide-based reagents are formed by coupling at least one body surface-binding peptide to at least one silica-binding peptide, either directly or through a molecular spacer. The part of the reagent comprising at least one body surface-binding peptide has affinity for the body surface, while the part of the reagent comprising at least one silica-binding peptide has affinity for silica and/or a silica-coated particulate benefit agent, thereby coupling the particulate benefit agent comprising an effective amount of silica to the body surface.

The coupling interaction may be a covalent bond or a non-covalent interaction, such as hydrogen bonding, electrostatic interaction, hydrophobic interaction, or Van der Waals interaction. In the case of a non-covalent interaction, the peptide-based reagents may be prepared by mixing at least one body surface-binding peptide and at least one silica-binding peptide and the optional spacer (if used) and allowing sufficient time for the interaction to occur. The unbound materials may be separated from the resulting peptide-based reagent using methods known in the art, for example, gel permeation chromatography.

The peptide-based reagents may also be prepared by covalently attaching at least one body surface-binding peptide to at least one of the present silica-binding peptides, either directly or through a spacer. Any known peptide or protein conjugation chemistry may be used to form the peptide-based reagents. Conjugation chemistries are well-known in the art (see for example, G. T. Hermanson, Bioconjuqate Techniques, 2^{nd} Ed., Academic Press, New York (2008)). Suitable coupling agents may include, but are not limited to, carbodiimide coupling agents, diacid chlorides, diisocyanates and other difunctional coupling reagents that are reactive toward terminal amine and/or carboxylic acid groups on the peptides. The preferred coupling agents are carbodiimide coupling agents, such as 1-ethyl-3-(3-dimethylaminopropyl)-carbodiimide (EDC) and N,N′-dicyclohexyl-carbodiimide (DCC), which may be used to activate carboxylic acid groups. Additionally, it may be necessary to protect reactive amine or carboxylic acid groups on the peptides to produce the desired structure for the peptide-based body surface reagent. The use of protecting groups for amino acids, such as t-butyloxycarbonyl (t-Boc), are well known in the art (see for example Stewart et al., supra; Bodanszky, supra; and Pennington et al., supra).

It may also be desirable to couple a body surface-binding peptide to a silica-binding peptide via a spacer/linker. The spacer serves to separate the binding peptide sequences to ensure that the binding affinity of the individual peptides is not adversely affected by the coupling. The spacer may also provide other desirable properties such as hydrophilicity, hydrophobicity, or a means for cleaving the peptide sequences to facilitate removal of the coloring agent.

The molecular “spacer” may also be any of a variety of molecules, such as alkyl chains, phenyl compounds, ethylene glycol, amides, esters and the like. In one embodiment, the organic spacers are hydrophilic and have a chain length from 1 to about 100 atoms, more preferably, from 2 to about 30 atoms. Examples of spacers may include, but are not limited to, ethanol amine, ethylene glycol, polyethylene with a chain length of 6 carbon atoms, polyethylene glycol with 3 to 6 repeating units, phenoxyethanol, propanolamide, butylene glycol, butylene glycolamide, propyl phenyl chains, and ethyl, propyl, hexyl, steryl, cetyl, and palmitoyl alkyl chains. The spacer may be covalently attached to the body surface-binding and iron oxide-based pigment-binding peptide sequences using any of the coupling chemistries described above. In order to facilitate incorporation of the spacer, a bifunctional cross-linking agent that contains a spacer and reactive groups at both ends for coupling to the peptides may be used. Suitable bifunctional cross-linking agents are well known in the art and may include, but are not limited to, diamines, such a as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyanates, such as hexamethylenediisocyanate; bis oxiranes, such as 1,4 butanediyl diglycidyl ether; dicarboxylic acids, such as succinyldisalicylate; and the like. Heterobifunctional cross-linking agents, which contain a different reactive group at each end, may also be used. Examples of heterobifunctional cross-linking agents may include, but are not limited to compounds having the following structure:

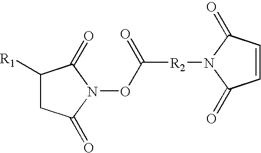

where: R₁ is H or a substituent group such as —SO₃Na, —NO₂, or —Br; and R₂ is a spacer such as —CH₂CH₂ (ethyl), —(CH₂)₃ (propyl), or —(CH₂)₃C₆H₅ (propyl phenyl). An example of such a heterobifunctional cross-linking agent is 3-maleimidopropionic acid N-hydroxysuccinimide ester. The N-hydroxysuccinimide ester group of these reagents reacts with amine groups on one peptide, while the maleimide group reacts with thiol groups present on the other peptide. A thiol group may be incorporated into the peptide by adding at least one cysteine group to at least one end of the binding peptide sequence (i.e., the C-terminus and/or the N-terminus). Several spacer amino acid residues, such as glycine, may be incorporated between the binding peptide sequence and the terminal cysteine to separate the reacting thiol group from the binding sequence. Moreover, at least one lysine residue may be added to at least one end of the binding peptide sequence to provide an amine group for coupling.

Additionally, the “spacer” may be a peptide spacer In addition, the peptide spacer may contain a specific enzyme cleavage site, such as the protease Caspase 3 site, given by SEQ ID NO: 252, which allows for the enzymatic removal of the silica-coated particulate benefit agent from the body surface. The peptide spacer may be from 1 to about 60 amino acids, preferably from 3 to about 50 amino acids in length. Examples of spacers include, but are not limited to, the sequences given by SEQ ID NOs: 253-261. When the peptide spacer is used to linker together two or more target-binding peptides to form a binding hand, the spacer will be referred to as a “peptide linker” that is preferably 3 to 50 amino acids in length. Examples peptide linkers are provided by SEQ ID NOs: 253-257. When the peptide spacer is used to couple a first region having affinity for a body surface and a second region having affinity for silica, the peptide spacer will be referred to as a “peptide bridge” what is preferably 1 to 60 amino acids in length. Examples of peptide bridges are provided as SEQ ID NOs: 258-261.

Peptide spacers may be linked to the binding peptide sequences by any method known in the art. For example, the entire peptide-based reagent may be prepared using the standard peptide synthesis methods described, supra. In addition, the binding peptides and peptide spacer may be combined using carbodiimide coupling agents (see for example, G. T. Hermanson, supra), diacid chlorides, diisocyanates and other difunctional coupling reagents that are reactive to terminal amine and/or carboxylic acid groups on the peptides, as described above. Alternatively, the entire peptide-based reagent may be prepared using the recombinant DNA and molecular cloning techniques described infra.

It may also be desirable to have multiple copies of the body surface-binding peptide(s) and/or the silica-binding peptide(s) coupled together to enhance the affinity between the peptide-based reagent and the body surface and/or the silica-coated particulate benefit agent. Multiple copies of the same body surface-binding peptide and/or silica-binding peptide or a combination of different body surface-binding peptides and silica-binding peptides may be used, so long as the composition comprises at least one of the present silica-binding peptides. The multi-copy peptide-based body surface reagents may comprise various spacers as described above. Examples of peptide-based reagents are provided as SEQ ID NOs: 29-34 and 298.

In one embodiment, the peptide-based reagent is a composition comprising at least one body surface-binding peptide (BSBP) and at least one of the present silica-binding peptides (SiBP), having the general structure [(BSBP)ₘ-(SiBP)ₙ]ₓ, where n and m independently range from 1 to about 10, preferably from 1 to about 5, and x may be 1 to about 10.

In another embodiment, the peptide-based reagent comprises a molecular spacer (S) separating the body surface-binding peptide from the silica-binding peptide, as described above. Multiple copies of the body surface-binding peptide(s) and/or the silica-binding peptide(s) may also be used and the multiple copies of the body surface-binding peptide and the silica-binding peptide may be separated from themselves and from each other by molecular spacers. In this embodiment, the peptide-based reagent is a composition comprising at least one body surface-binding peptide, at least one spacer (S), and at least one of the present silica-binding peptides, having the general structure [[(BSBP)ₘ-S_{q}]ₓ-[(SiBP)ₙ-Sᵣ]_{z}]_{y}, where n, m, x, and z independently range from 1 to about 10, y is from 1 to about 5, and where q and r are each independently 0 or 1, provided that both q and r are not 0. In one embodiment, m and n independently range from 1 to about 5, and x and z independently range from 1 to about 3.

In another embodiment, the body surface-binding peptide is a hair-binding peptide and the peptide-based reagent is a composition comprising at least one hair-binding peptide (HBP) and at least one of the present silica-binding peptides (SiBP), having the general structure [(HBP)ₘ-(SiBP)ₙ]ₓ where n and m independently range from 1 to about 10, preferably from 1 to about 5, and x may be 1 to about 10.

In another embodiment, the body surface-binding peptide is a hair-binding peptide and the peptide-based body reagent is a composition comprising at least one hair-binding peptide (HBP), at least one spacer (S), and at least one of the present silica-binding peptides (SiBP), having the general structure [[(HBP)ₘ-S_{q}]ₓ-[(SiBP)ₙ-Sᵣ]_{z}]_{y}, where n, m, x, and z independently range from 1 to about 10, y is from 1 to about 5, and where q and r are each independently 0 or 1, provided that both q and r are not 0. In one embodiment, m and n independently range from 1 to about 5, and x and z independently range from 1 to about 3.

In another embodiment, the body surface-binding peptide is a skin-binding peptide and the peptide-based body surface coloring reagent is a composition comprising at least one skin-binding peptide (SBP) and at least one of the present silica-binding peptides (SiBP), having the general structure [(SBP)ₘ-(SiBP)ₙ]ₓ, where n and m independently range from 1 to about 10, preferably from 1 to about 5, and x may be 1 to about 10.

In another embodiment, the body surface-binding peptide is a skin-binding peptide and the peptide-based reagent is a composition comprising at least one skin-binding peptide (SBP), at least one spacer (S), and at least one of the present silica-binding peptides (SiBP), having the general structure [[(SBP)ₘ-S_{q}]ₓ-[(SiBP)ₙ-Sᵣ]_{z}]_{y}, where n, m, x, and z independently range from 1 to about 10, y is from 1 to about 5, and where q and r are each independently 0 or 1, provided that both q and r are not 0. In one embodiment, m and n independently range from 1 to about 5, and x and z independently range from 1 to about 3.

In another embodiment, the body surface-binding peptide is a nail-binding peptide and the peptide-based reagent is a composition comprising at least one nail-binding peptide (NBP) and at least one of the present silica-binding peptides (SiBP), having the general structure [(NBP)ₘ-(SiBP)ₙ]ₓ where n and m independently range from 1 to about 10, preferably from 1 to about 5, and x may be 1 to about 10.

In another embodiment, the body surface-binding peptide is a nail-binding peptide and the peptide-based reagent is a composition comprising at least one nail-binding peptide (NBP), at least one spacer (S), and at least one of the present silica-binding peptides (SiBP), having the general structure [[(NBP)ₘ-S_{q}]ₓ-[(SiBP)ₙ-Sᵣ]_{z}]_{y}, where n, m, x, and z independently range from 1 to about 10, y is from 1 to about 5, and where q and r are each independently 0 or 1, provided that both q and r are not 0. In one embodiment, m and n independently range from 1 to about 5, and x and z independently range from 1 to about 3.

In another embodiment, the body surface-binding peptide is a tooth-binding peptide and the peptide-based reagent is a composition comprising at least one tooth-binding peptide (TBP) and at least one of the present silica-binding peptides (SiBP), having the general structure [(TBP)ₘ-(SiBP)ₙ]ₓ where n and m independently range from 1 to about 10, preferably from 1 to about 5, and x may be 1 to about 10.

In another embodiment, the body surface-binding peptide is a tooth-binding peptide and the peptide-based reagent is a composition comprising at least one tooth-binding peptide (TBP), at least one spacer (S), and at least one of the present silica-binding peptides (SiBP), having the general structure [[(TBP)ₘ-S_{q}]ₓ-[(SiBP)ₙ-Sᵣ]_{Z}]_{y}, where n, m, x, and z independently range from 1 to about 10, y is from 1 to about 5, and where q and r are each independently 0 or 1, provided that both q and r are not 0. In one embodiment, m and n independently range from 1 to about 5, and x and z independently range from 1 to about 3.

In a further embodiment, the peptide-based reagent comprises one or more of the present silica-binding peptides selected form the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, and 27; and at least one of the tooth-binding peptides selected from the group consisting of SEQ ID NOs: 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 295, 296 and 297.

It should be understood that as used herein BSBP, HBP, SBP, NBP, TBP, and SiBP are generic designations and are not meant to refer to a single body surface-binding peptide, hair-binding peptide, skin-binding peptide, nail-binding peptide, tooth-binding or silica-binding peptide sequence, respectively. Where m or n as used above, is greater than 1, it is well within the scope of the invention to provide for the situation where a series of body surface-binding peptides of different sequences and silica-binding peptides of different sequences (i.e., at least one of the present silica-binding peptides) may form a part of the composition. Additionally, S is a generic term and may refer to more than one single spacer. Where x and y are greater than 1, it is well within the scope of the invention to provide for the situation where a series of different spacers may form a part of the composition.

### Production of Peptides

The present peptides and peptide-based reagents may be prepared using standard peptide synthesis methods (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the silica-binding peptides as well as the peptide-based reagents (particularly when the entire peptide-based reagent is produced as a single amino acid chain) may be prepared using recombinant DNA and molecular cloning techniques. Genes encoding the peptides may be produced in heterologous host cells, particularly in the cells of microbial hosts.

Preferred heterologous host cells for expression of the binding peptides of the present invention are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Yarrowia, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the peptides. Such vectors include, but are not limited to, chromosomal, episomal and virus-derived vectors, such as vectors derived from bacterial plasmids, from bacteriophage, from transposons, from insertion elements, from yeast episomes, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the binding peptides. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the binding peptides of the present invention including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence, as well as an appropriate promoter or control sequence, may be employed to transform an appropriate host to permit the host to express the peptide of interest. Cell-free translation systems can also be employed to produce such peptides using RNAs derived from the DNA constructs. Optionally, it may be desired to produce the gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are known in the art (see for example EP 546049 and WO 93/24631). The secretion signal DNA or facilitator may be located between the expression-controlling DNA and gene or gene fragment, and in the same reading frame with the latter.

### Personal Care Compositions

The peptides and peptide-based reagents may be used in personal care compositions in conjunction with a silica-based (such as silica-coated) particulate benefit agents (such as a silica-coated pigments) to provide a benefit to a body surface. The body surface-binding peptide portion of the peptide-based agent has an affinity for the body surface, while the silica-binding peptide portion has an affinity for the silica-coated particulate benefit agent. The peptide-based reagent may be present in the same composition as the silica-coated particulate benefit agent (e.g., silica-coated pigment) or the peptide-based reagent and the silica-coated particulate benefit agent (e.g., silica-coated pigment) may be present in two different personal care compositions that are applied to the body surface in any order, as described below. Personal care compositions include, but are not limited to, hair care/coloring compositions, skin care/coloring compositions, cosmetic compositions, nail polish compositions, and oral care/tooth coloring compositions.

### Hair Care Compositions

In one embodiment, the peptide-based reagent is a component of a hair care composition and the peptide-based reagent comprises at least one hair-binding peptide and at least one of the present silica-binding peptides. Hair care compositions are herein defined as compositions for the treatment of hair including, but not limited to, shampoos, conditioners, rinses, lotions, aerosols, gels, and mousses. An effective amount of the peptide-based reagent for use in hair care compositions is a concentration of about 0.01% to about 10%, preferably about 0.01% to about 5% by weight relative to the total weight of the composition. This proportion may vary as a function of the type of hair care composition. Additionally, the hair care composition may further comprise at least one pigment in addition to a silica-coated pigment or may comprise a mixture of two or more silica-coated pigments.

The concentration of the peptide-based reagent in relation to the concentration of the silica-coated particulate benefit agent may need to be optimized for best results. Additionally, a mixture of different peptide-based reagents having an affinity for one or more additional silica-coated particulate benefit agents may be used in the composition to obtain the desired benefit, such as color. The peptide-based reagents in the mixture need to be chosen so that there is little or no interaction between the peptide-based reagents that may adversely impact the desired beneficial effect (such as coloring). Suitable mixtures of peptide-based reagents may be determined by one skilled in the art using routine experimentation. If a mixture of peptide-based reagents is used in the composition, the total concentration of the reagents is about 0.01% to about 10% by weight relative to the total weight of the composition.

The composition may further comprise a cosmetically-acceptable medium for hair care compositions, examples of which are described by Philippe et al. in U.S. Pat. No. 6,280,747; by Omura et al. in U.S. Pat. No. 6,139,851; and Cannell et al. in U.S. Pat. No. 6,013,250; each of which is incorporated herein by reference in their entirety. For example, the hair care compositions can be aqueous, alcoholic or aqueous-alcoholic solutions, the alcohol preferably being ethanol or isopropanol, in a proportion of from about 1 to about 75% by weight relative to the total weight for the aqueous-alcoholic solutions. Additionally, the hair care compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes.

### Hair Coloring Compositions

The peptide-based reagent may be a component of a hair coloring composition. Hair coloring compositions are compositions for the coloring or dyeing of hair, which comprise one or more coloring agents and at least one of the present peptides. Coloring agents as herein defined are comprised of at least one silica-coated pigment and may further include any dye, additional pigment(s), and the like that may be used to change the color of a body surface, such as hair, skin, nails, or teeth. Hair coloring agents are well known in the art (see for example Green et al. supra, CFTA International Color Handbook, 2^{nd} ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany).

An effective amount of a peptide-based reagent for use in a hair coloring composition is herein defined as a proportion of from about 0.01% to about 20% by weight relative to the total weight of the composition. Additionally, a mixture of different peptide-based reagents having an affinity for different silica-coated pigments may be used in the composition. The peptide-based reagents in the mixture need to be chosen so that there is no adverse interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.01% to about 20% by weight relative to the total weight of the composition.

Components of a cosmetically-acceptable medium for hair coloring compositions are described by Dias et al. in U.S. Pat. No. 6,398,821 and by Deutz et al. in U.S. Pat. No. 6,129,770; each of which is incorporated herein by reference in its entirety. For example, hair coloring compositions may contain sequestrants, stabilizers, thickeners, buffers, carriers, surfactants, solvents, antioxidants, polymers, and conditioners.

### Skin Care Compositions

The peptide-based reagent may be a component of a skin care composition. Skin care compositions are compositions for the treatment of skin including, but not limited to, skin care, skin cleansing, make-up, and anti-wrinkle products. An effective amount of the peptide-based reagent for use in a skin care composition is a concentration of about 0.01% to about 10%, preferably about 0.01% to about 5% by weight relative to the total weight of the composition. This proportion may vary as a function of the type of skin care composition. Additionally, a mixture of different peptide-based reagents having an affinity for different (additional) silica-coated particulate benefit agents (such as silica-coated pigments) may be used in the composition. The peptide-based reagents in the mixture need to be chosen so that there is no interaction between the peptides that mitigates the beneficial effect to the skin. Suitable mixtures of peptide-based reagents may be determined by one skilled in the art using routine experimentation. If a mixture of peptide-based reagents is used in the composition, the total concentration of the reagents is about 0.01% to about 10% by weight relative to the total weight of the composition. The skin care composition may further comprise in addition to a silica-coated particulate benefit agent (e.g., a silica-coated pigment) at least one additional pigment, suitable examples of which are given above. The concentration of the peptide-based reagent in relation to the concentration of the silica-coated particulate benefit agent (e.g., silica-coated pigment) may need to be optimized for best results.

The skin care composition may further comprise a cosmetically acceptable medium for skin care compositions, examples of which are described by Philippe et al., supra. The cosmetically acceptable medium may be an anhydrous composition containing a fatty substance in a proportion generally of from about 10 to about 90% by weight relative to the total weight of the composition, where the fatty phase contains at least one liquid, solid or semi-solid fatty substance. The fatty substance may include, but is not limited to, oils, waxes, gums, and so-called pasty fatty substances. Alternatively, the skin care compositions may be in the form of a stable dispersion such as a water-in-oil or oil-in-water emulsion. Additionally, the skin care compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes.

### Skin Coloring Compositions

The peptide-based reagent may be a component of a skin coloring composition. The skin coloring composition may comprises one or more coloring agents in addition to at least one silica-based (e.g., silica-coated) pigment.

The skin coloring compositions may be any cosmetic or make-up product, including but not limited to, foundations, blushes, lipsticks, lip liners, lip glosses, eyeshadows and eyeliners. These may be anhydrous make-up products comprising a cosmetically acceptable medium which contains a fatty substance, or they may be in the form of a stable dispersion such as a water-in-oil or oil-in-water emulsion, as described above. In these compositions, an effective amount of the peptide-based reagent is generally from about 0.01% to about 40% by weight relative to the total weight of the composition. Additionally, a mixture of different peptide-based reagents having an affinity for different silica-based pigments may be used in the composition. The peptide-based reagents in the mixture need to be chosen so that there is no interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.01% to about 40% by weight relative to the total weight of the composition.

### Cosmetic Compositions

The peptide-based reagent may be a component of a cosmetic composition comprising at least one silica-based (e.g. silica-coated). Cosmetic compositions may be applied to the eyelashes or eyebrows including, but not limited to mascaras, and eyebrow pencils. The cosmetic compositions may comprise one or more coloring agents in addition to at least one silica-coated pigment or may be a mixture of silica-coated pigments.

An effective amount of a peptide-based reagent for use in a cosmetic composition is a proportion of from about 0.01% to about 20% by weight relative to the total weight of the composition. Additionally, a mixture of different peptide-based reagents having affinity for different silica-coated pigments may be used in the composition. The peptide-based reagents in the mixture need to be chosen so that there is no adverse interaction between the peptides that mitigates the beneficial effect. Suitable mixtures of peptide-based reagents may be determined by one skilled in the art using routine experimentation. If a mixture of peptide-based body surface coloring reagents is used in the composition, the total concentration of the reagents is about 0.01% to about 20% by weight relative to the total weight of the composition.

Cosmetic compositions may be anhydrous make-up products comprising a cosmetically acceptable medium which contains a fatty substance in a proportion generally of from about 10 to about 90% by weight relative to the total weight of the composition, where the fatty phase containing at least one liquid, solid or semi-solid fatty substance, as described above. The fatty substance may include, but is not limited to, oils, waxes, gums, and so-called pasty fatty substances. Alternatively, these compositions may be in the form of a stable dispersion such as a water-in-oil or oil-in-water emulsion, as described above.

### Nail Polish Compositions

The peptide-based reagent may be a component of a nail polish composition. The nail polish compositions may be used for coloring fingernails and toenails. The nail polish compositions comprise at least one peptide-based reagent and at least one silica-coated pigment. The nail polish compositions may contain one or more additional coloring agents.

An effective amount of a peptide-based reagent for use in a nail polish composition is a proportion of from about 0.01% to about 20% by weight relative to the total weight of the composition. Additionally, a mixture of different peptide-based reagents having affinity for different silica-based pigments may be used in the composition. The peptide-based in the mixture need to be chosen so that there is no adverse interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.01% to about 20% by weight relative to the total weight of the composition.

Components of a cosmetically-acceptable medium for nail polish compositions are known in the art. Examples of cosmetically-acceptable mediums for nail polish compositions are described by Philippe et al., supra. The nail polish composition typically contains a solvent and a film forming substance, such as cellulose derivatives, polyvinyl derivatives, acrylic polymers or copolymers, vinyl copolymers and polyester polymers. Additionally, the nail polish may contain a plasticizer, such as tricresyl phosphate, benzyl benzoate, tributyl phosphate, butyl acetyl ricinoleate, triethyl citrate, tributyl acetyl citrate, dibutyl phthalate or camphor.

### Oral Care Compositions

The peptide-based reagent may be a component of an oral care composition. Contemplated herein are oral care compositions comprising an effective amount of at least one of the present peptide-based reagents and an effective amount of at least one particulate benefit agent. As used here, the term “effective amount” is that amount of at least one of the present peptide compositions or that amount of at least one or more particles comprising particulate benefit agent incorporated into the oral care composition to achieve the desired benefit such as, for example, tooth whitening.

The oral care compositions may be in the form of powder, paste, gel, liquid, ointment, or tablet. Exemplary oral care compositions may include, but are not limited to, toothpaste, dental cream, gel or tooth powder, mouth wash, breath freshener, gum, candy, and dental floss. The oral care compositions comprise an effective amount of a peptide-based reagent and at least one silica-coated particulate benefit agent in an orally-acceptable carrier medium. An effective amount of a peptide-based reagent for use in an oral care composition may vary depending on the type of product. Typically, the effective amount of the peptide-based reagent is a proportion from about 0.01% to about 90% by weight relative to the total weight of the composition. Additionally, a mixture of different peptide-based reagents having affinity for different silica-coated particulate benefit agents (such as silica-coated pigments) may be used in the oral care composition. The peptide-based reagents in the mixture need to be chosen so that there is no adverse interaction between the peptides that mitigates the beneficial effect. Suitable mixtures of peptide-based reagents may be determined by one skilled in the art using routine experimentation. If a mixture of peptide-based reagents is used in the composition, the total concentration of the reagents is about 0.001% to about 90% by weight relative to the total weight of the composition.

Components of an orally-acceptable carrier medium are known in the art. Examples of orally-acceptable components are described by White et al. in U.S. Pat. No. 6,740,311; Lawler et al. in U.S. Pat. No. 6,706,256; and Fuglsang et al. in U.S. Pat. No. 6,264,925; each of which is incorporated herein by reference in its entirety. For example, the oral care composition may comprise one or more of the following: abrasives, surfactants, chelating agents, fluoride sources, thickening agents, buffering agents, solvents, humectants, carriers, bulking agents, and oral benefit agents, such as enzymes, anti-plaque agents, anti-staining agents, anti-microbial agents, anti-caries agents, flavoring agents, coolants, salivating agents, gingivitis treatment agents, periodontitis treatment agents, and combinations thereof.

In one embodiment, the peptide-based reagent may be used to detect the presence of a material of interest on a tooth (e.g., the use of a peptide-based reagent for diagnostic applications). For example, the peptide-based reagent may be used to detect the presence of a pellicle coating on teeth immediately after an abrasive cleaning/polishing procedure (e.g., a dental office cleaning/polishing procedure).

An oral care benefit agent may be encapsulated or absorbed in a silica-coated porous particle or a hollow porous silica shell particle for delivery of the desired benefit agent. Hollow porous silica particles suitable for delivery of an encapsulated or absorbed benefit agent may be prepared by using any number of well known methods (see U.S. Pat. No. 5,024,826 to Linton, H.; and U.S. Pat. No. 6,221,326 to Amiche, F., each herein incorporated by reference in its entirety). The porous silica shells typically have an average particle size ranging from 20 nm to 15 μm, a pore size ranging from 3 nm to 10 nm, a shell thickness ranging from 2 nm to 50 nm, and a specific surface of 25±400 m²/g.

### Tooth Whitening Compositions

Oral care compositions may comprise at least one orally-acceptable silica-coated white colorant suitable for whitening teeth. Suitable white colorants which may be used in the oral care composition may include, but are not limited to, white pigments such as titanium dioxide, titanium dioxide nanoparticles and zinc oxide, white minerals such as hydroxyapatite, Zircon (zirconium silicate), and mixtures thereof. However, it may be desirable to further include at least one non-white pigment in an oral care composition to achieve the desired coloration. Methods to apply an effective amount of a silica coating to a particle are well-known in the art (see, for example, U.S. Pat. No. 2,885,366 to Iler). In a preferred embodiment, the white colorant comprises silica-coated titanium dioxide.

### Methods for Coupling a Silica-Coated Particulate Benefit Agent to a Body Surface

The peptide-based reagents may be used in conjunction with a silica-coated particulate benefit agent to provide a benefit to body surfaces, such as hair, skin, nails, and teeth. The peptide-based reagent may be present in the same composition as the silica-coated particulate benefit agent, or the peptide-based reagent and the silica-coated particulate benefit agent may be present in two different compositions. In one embodiment, a personal care composition comprising at least one peptide-based reagent and at least one silica-coated particulate benefit agent is applied to a body surface for a time sufficient for the peptide-based reagent, which is non-covalently coupled to the silica-coated particulate benefit agent via the silica-binding peptide, to bind to the body surface. In another embodiment, at least one silica-coated particulate benefit agent is applied to a body surface prior to the application of at least one of the present peptide-based reagents. In another embodiment, a composition comprising at least one of the present peptide-based reagents is applied to the body surface prior to the application of at least one silica-coated particulate benefit agent. In another embodiment, at least one silica-coated particulate benefit agent and at least one of the present peptide-based reagents are applied to the body surface concomitantly. Optionally, the composition comprising the peptide-based reagent may be reapplied to the body surface after the application of the silica-coated particulate benefit agent and the initial application of the composition comprising the peptide-based reagent.

In any of the methods described above, a composition comprising a polymeric sealant may be applied to the body surface after the application of at least one silica-coated benefit agent and the composition comprising at least one peptide-based reagent in order to further enhance the durability of the benefit. The polymeric sealant may be present in the composition at a concentration of about 0.25% to about 10% by weight relative to the total weight of the composition. Polymeric sealants are well-known in the art of personal care products and may include, but are not limited to, poly(allylamine), acrylates, acrylate copolymers, polyurethanes, carbomers, methicones, amodimethicones, polyethylenene glycol, beeswax, siloxanes, and the like. The choice of polymeric sealant depends on the particular silica-coated particulate benefit agent and the peptide-based reagent. The optimum amount of polymeric sealant may be readily determined by one skilled in the art using routine experimentation.

### Methods for Coloring Hair

The peptide-based reagents may be used to attach a silica-coated pigment to the surface of hair. The peptide-based body surface coloring reagent and the silica-coated pigment may be applied to the hair from any suitable hair care composition, for example a hair colorant, a hair shampoo or a hair conditioner composition. These hair care compositions are well-known in the art and suitable compositions are described above.

In one embodiment, a silica-coated pigment is applied to the hair for a time sufficient for the silica-coated pigment to bind to the hair, typically between about 5 seconds to about 60 minutes. Optionally, the hair may be rinsed to remove the silica-coated pigment that has not bound to the hair. Then, a composition comprising a peptide-based reagent is applied to the hair for a time sufficient for the peptide-based reagent to bind to the hair and the silica-coated pigment, typically between about 5 seconds to about 60 minutes. The composition comprising the peptide-based reagent may be rinsed from the hair or left on the hair.

A composition comprising a peptide-based reagent may be applied to the hair for a time sufficient for the hair-binding peptide block of the peptide-based reagent to bind to the hair, typically between about 5 seconds to about 60 minutes. Optionally, the hair may be rinsed to remove the composition comprising the peptide-based reagent that has not bound to the hair. Then, a silica-coated pigment is applied to the hair for a time sufficient for the silica-coated pigment to bind to the silica-binding portion of the peptide-based reagent, typically between about 5 seconds to about 60 minutes. The unbound silica-coated pigment may be rinsed from the hair or left on the hair.

A silica-coated pigment and peptide-based reagent may be applied to the hair concomitantly for a time sufficient for the peptide-based reagent to bind to hair and the silica-coated pigment, typically between about 5 seconds to about 60 minutes. Optionally, the hair may be rinsed to remove the unbound silica-coated pigment and the composition comprising a peptide-based reagent from the hair.

In one embodiment, a silica-coated pigment may be provided as part of a composition comprising a peptide-based reagent, for example a hair coloring composition. A composition comprising the silica-coated pigment, such as a silica-coated iron oxide pigment and/or a silica-coated titanium dioxide pigment, and the peptide-based reagent may be applied to the hair for a time sufficient for the peptide-based reagent, which is coupled to the silica-coated pigment through the silica-binding peptide block, to bind to the hair, typically between about 5 seconds to about 60 minutes. The composition comprising the silica-coated pigment and the peptide-based reagent may be rinsed from the hair or left on the hair.

In any of the methods described above, the hair care composition comprising a peptide-based reagent may be reapplied to the hair after the application of the silica-coated pigment and the initial application of the composition comprising a peptide-based reagent in order to further enhance the durability of the colorant.

Additionally, in any of the methods described above, a composition comprising a polymeric sealant may be applied to the hair after the application of the silica-coated pigment and the composition comprising a peptide-based reagent in order to further enhance the durability of the colorant. The composition comprising the polymeric sealant may be an aqueous solution or a hair care composition, such as a conditioner or rinse, comprising the polymeric sealant. The polymeric sealant may be present in the composition at a concentration of about 0.25% to about 10% by weight relative to the total weight of the composition. Polymeric sealants are well know in the art of personal care products and may include, but are not limited to, poly(allylamine), acrylates, acrylate copolymers, polyurethanes, carbomers, methicones, amodimethicones, polyethylenene glycol, beeswax, siloxanes, and the like. The choice of polymeric sealant may depend on the particular silica-coated pigment and the peptide-based reagent used. The optimum polymeric sealant may be readily determined by one skilled in the art using routine experimentation.

### Methods for Coloring Skin

The peptide-based reagents may be used to attach a silica-coated pigment to a skin surface, thereby coloring the skin. The peptide-based reagent and the silica-coated pigment may be applied to the skin from any suitable skin care composition, for example a skin colorant or skin conditioner composition. These skin care compositions are well-known in the art and suitable compositions are described above.

A silica-coated pigment may be applied to skin for a time sufficient for the silica-coated pigment to bind to the skin surface, typically between about 5 seconds to about 60 minutes. The skin may be rinsed to remove the silica-coated pigment that has not bound to the skin. Then, a composition comprising at least one of the present peptide-based reagents is applied to the skin surface for a time sufficient for the peptide-based reagent to bind to the skin surface and the silica-coated pigment, typically between about 5 seconds to about 60 minutes. The composition comprising the peptide-based reagent may be rinsed from the skin or left on the skin.

A composition comprising a peptide-based reagent may be applied to the skin for a time sufficient for the skin-binding peptide block of the peptide-based reagent to bind to the skin surface, typically between about 5 seconds to about 60 minutes. The skin may be rinsed to remove the composition that has not bound to the skin. Then, a silica-coated pigment, such as a silica-coated iron oxide-based pigment or a silica-coated titanium dioxide pigment, may be applied to the skin for a time sufficient for the silica-coated pigment to bind to the silica-binding portion of the peptide-based reagent, typically between about 5 seconds to about 60 minutes. The unbound silica-coated pigment may be rinsed from the skin or left on the skin.

A silica-coated pigment and a composition comprising at least one of the present peptide-based reagents may be applied to the skin concomitantly for a time sufficient for the peptide-based reagent to bind to skin and the silica-coated pigment, typically between about 5 seconds to about 60 minutes. The skin may be rinsed to remove the unbound silica-coated pigment and the composition comprising a peptide-based reagent from the skin.

A silica-coated pigment may be provided as part of the composition comprising at least one of the present peptide-based reagents, for example a skin coloring composition. The composition comprising the silica-coated pigment and at least one of the present peptide-based reagent may be applied to the skin for a time sufficient for the peptide-based reagent, which is coupled to the silica-coated pigment through the silica-binding portion, to bind to the skin surface, typically between about 5 seconds to about 60 minutes. The composition comprising the silica-coated pigment and the peptide-based coloring reagent may be rinsed from the skin or left on the skin.

In any of the methods described above, the composition comprising a peptide-based reagent may be reapplied to the skin after the application of the silica-coated pigment and the initial application of the composition comprising at least one peptide-based reagent in order to further enhance the durability of the silica-coated pigment.

Additionally, in any of the methods described above, a composition comprising a polymeric sealant may be applied to the skin after the application of the silica-coated pigment and the peptide-based reagent in order to further enhance the durability of the colorant. Any of the polymeric sealants described above for hair coloring may be used in the form of an aqueous solution or a skin care composition.

### Methods for Coloring Nails, Eyebrows, and Eyelashes

The methods described above for coloring hair and skin may also be applied to coloring finger nails, toenails, eyebrows, and eyelashes, by applying the appropriate composition, specifically, a nail polish composition or a cosmetic composition to the body surface of interest.

### Methods for Whitening Teeth

The peptide-based reagents may be used in conjunction with at least one silica-coated white colorant to whiten one or more teeth (i.e., at least one tooth targeted for whitening). The tooth-binding peptide portion of the peptide-based reagent has an affinity for a tooth surface, while the silica-binding peptide portion has an affinity for a silica-coated white colorant (such as a silica-coated white pigment). The peptide-based tooth whitening reagent may be present in the same composition as the white silica-coated colorant, or the peptide-based tooth whitening reagent and the silica-coated white colorant may be present in two different compositions. In one embodiment, an oral care composition comprising at least one peptide-based reagent and at least one silica-coated white colorant may be applied to a tooth surface for a time sufficient for the peptide-based reagent, which is non-covalently coupled to the silica-coated white colorant via the silica-binding peptide portion, to bind to the tooth surface. In another embodiment, at least one silica-coated white colorant may be applied to a tooth surface prior to the application of a composition comprising at least one peptide-based reagent. In another embodiment, a composition comprising at least one peptide-based reagent may be applied to the tooth surface prior to the application of the silica-coated white colorant. In another embodiment, at least one silica-coated white colorant and a composition comprising at least one peptide-based reagent may be applied to the tooth surface concomitantly, that is together. The composition comprising the peptide-based reagent may be reapplied to the tooth surface after the application of the silica-coated white colorant and the initial application of the composition comprising the peptide-based reagent.

In one embodiment, a composition comprising a peptide-based reagent may be applied to a tooth surface for a time sufficient for the tooth-binding peptide block of the peptide-based whitening reagent to bind to the teeth, typically between about 5 seconds to about 60 minutes. The tooth surface may be rinsed to remove the composition that has not bound to the tooth surface. Then, at least one silica-coated white colorant is applied to the tooth for a time sufficient for the at least one silica-coated white colorant to bind to the silica-binding block of the peptide-based reagent, typically between about 5 seconds to about 60 minutes. The unbound silica-coated white colorant may be rinsed from the teeth or left on the teeth.

In another embodiment, at least one silica-coated white colorant and a composition comprising at least one peptide-based reagent may be applied to at least one tooth concomitantly (i.e., at the same time)) for a period of time sufficient for the peptide-based reagent to bind to the tooth surface and the silica-coated white colorant, typically between about 5 seconds to about 60 minutes. The tooth surface may be rinsed to remove the unbound silica-coated white colorant and the composition comprising a peptide-based reagent from the tooth surface.

In another embodiment, at least one silica-coated white colorant may be provided as part of a composition comprising at least one peptide-based reagent, such as a tooth whitening composition. The composition comprising the silica-coated white colorant and the peptide-based reagent may be applied to at least one tooth for a period of time sufficient for the peptide-based reagent, which is coupled to the silica-coated white colorant through the silica-binding peptide portion, to bind to the tooth surface, typically between about 5 seconds to about 60 minutes. The composition comprising the silica-coated white colorant and the peptide-based reagent may be rinsed from the tooth surface or left on the tooth surface.

In any of the methods described above, the composition comprising at least one of the present peptide-based reagents may be reapplied to the tooth surface after the application of the silica-coated white colorant and the initial application of the composition comprising the peptide-based reagent in order to further enhance the durability of the colorant.

Additionally, in any of the methods described above, a composition comprising an additional polymeric sealant may be applied to the tooth surface after the application of the silica-coated white colorant and the composition comprising at least one of the present peptide-based reagents in order to further enhance the durability of the colorant. The composition comprising the additional polymeric sealant may be an aqueous solution or an oral care composition, such as a toothpaste or mouthwash, comprising the additional polymeric sealant (such as GANTREZ® copolymers). Typically, the polymeric sealant is present in the composition at a concentration of about 0.25% to about 10% by weight relative to the total weight of the composition. Additional polymeric sealants well know in the art of personal care products may include, but are not limited to, poly(allylamine), acrylates, acrylate copolymers, polyurethanes, carbomers, methicones, amodimethicones, polyethylenene glycol, beeswax, siloxanes, PLASDONE® and POLYPLASDONE® (linear and cross-linked polyvinylpyrrolidone homopolymers, respectively; available for International Specialty Products), and the like. The choice of polymeric sealant depends on the particular silica-coated white colorant and the peptide-based reagent used. The optimum polymeric sealant may be readily determined by one skilled in the art using routine experimentation.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “mg” means milligram(s), “g” means the gravitation constant, “rpm” means revolution(s) per minute, “pfu” means plaque forming unit(s), “BSA” means bovine serum albumin, “ELISA” means enzyme linked immunosorbent assay, “IPTG” means isopropyl β-D-thiogalactopyranoside, “A” means absorbance, “A₄₅₀” means the absorbance measured at a wavelength of 450 nm, “TBS” means Tris-buffered saline, “TBST-X” means Tris-buffered saline containing TWEEN® 20 where “X” is the weight percent of TWEEN® 20, “Xgal” means 5-bromo-4-chloro-3-indolyl-beta-D-galactopyranoside, “SEM” means standard error of the mean, “MW” means molecular weight, “M_{w}” means weight-average molecular weight, “vol %” means volume percent, and “wt %” means weight percent.

### General Methods

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989. All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma-Aldrich Chemical Company (St. Louis, Mo.), unless otherwise specified.

### Example 1

### Selection of Peptides that Bind to Silica-Coated Titanium Dioxide (TiO₂)) Particles Using Standard Biopanning

The purpose of this example was to identify phage peptides that bind to silica coated particles using standard phage display biopanning method.

Silica-coated particles were produced by DuPont (Titanium Dioxide with 3% silica coatings). The average size of the primary particles was around 0.61 microns in diameter. The silica-coated particles were incubated in SUPERBLOCK® blocking buffer (Pierce Chemical Company, Rockford, Ill.; Prod. #37535) for 1 hour at room temperature (−22° C.), followed by 3 washes with TBST (TBS in 0.5% TWEEN® 20). Libraries of phage containing random peptide inserts (10¹¹ pfu) from 7 to 20 amino acids were added to each tube. After 60 minutes of incubation at room temperature and shaking at 50 rpm, unbound phage were removed by aspirating the liquid out of each well followed by 6 washes with 1.0 mL TBS containing the detergent TWEEN® 20 (TBST, T-0.5%) and 30% of NEUTROGENA® shampoo (NEUTROGENA® Clean Replenishing Moisturizing Shampoo, Neutrogena Corporation, Los Angeles, Calif.).

The particle samples were then transferred to a clean tube, and 200 μL of elution buffer consisting of 1 mg/mL BSA in 0.2 M glycine-HCl, pH 2.2, was added to each well and incubated for 10 min to elute the bound phages. Then, 32 μL of neutralization buffer consisting of 1 M Tris-HCl, pH 9.2, was added to each tube. The phage particles, which were in the elution buffer as well as on the particles, were amplified by incubating with diluted E. coli ER2738 cells, from an overnight culture diluted 1:100 in LB medium, at 37° C. for 4.5 h. After this time, the cell culture was centrifuged for 30 seconds and the upper 80% of the supernatant was transferred to a fresh tube and then 1/6 volume of PEG/NaCl (20% polyethylene glyco-800, 2.5 M sodium chloride) was added, and the phage was allowed to precipitate overnight at 4° C. The precipitate was collected by centrifugation at 10,000×g at 4° C. and the resulting pellet was resuspended in 1 mL of TBS. This was the first round of amplified stock. The amplified first round phage stock was then tittered. For the 2nd, 3rd and 4^{th} round of biopanning, more than 2×10¹¹ pfu of phage stock from the previous round was used. The biopanning process was repeated under the same conditions as described above.

After the 4^{th} round of biopanning, 95 random single phage plaque lysates were prepared following the manufacture's instructions (New England BioLabs, Beverly, Mass.) and the single stranded phage genomic DNA was purified using the QIAprep Spin M13 Kit (Qiagen, Valencia, Calif.) and sequenced at the DuPont Sequencing Facility using −96 gill sequencing primer (5′-CCCTCATAGTTAGCGTAACG-3′; SEQ ID NO: 262). The displayed peptide is located immediately after the signal peptide of gene III. Based on the peptide sequences, 30 phage candidates that showed significant enrichment were selected for further silica-coated particle binding analysis. The amino acid sequences of selected phage candidates are listed in Table 1.

TABLE 1
Amino Acid Sequences of Silica-Coated Titanium
Dioxide (TiO₂) Particle Binding Candidates.

| | Amino Acid Sequence | |
|---|---|---|
| Phage ID | Peptides that Bind to Silica | |
| No. | Coated TiO₂ Particles | SEQ ID NO. |
| Soti-1 | AEAKRHPVVPLHEQHGHHEL | 1 |
| Soti-2 | APQTWNRPHPGHPNVHTR | 2 |
| Soti-3 | ATTPPSGKAAAHSAARQKGN | 3 |
| Soti-4 | DGRPDNPKHQQSYNRQLPRQ | 4 |
| Soti-5 | DHNNRQHAVEVRENKTHTAR | 5 |
| Soti-6 | GPEPRALNPKRHMDPATQIR | 6 |
| Soti-7 | HDHHQTHNVLHGMKK | 7 |
| Soti-8 | HHDRAEPRGMAATLAQTI | 8 |
| Soti-9 | HHNHMTGADNPIFHNNTAHR | 9 |
| Soti-10 | HNHAQMLRPEPTGISHKN | 10 |
| Soti-11 | HTNDNGQSTPRRDPPAFQRK | 11 |
| Soti-12 | HTNHHYDQKMHGPLPTPY | 12 |
| Soti-13 | LNSMSDKHHGHQNTATRNQH | 13 |
| Soti-14 | MHKPNNPDTHRSTPSPLGKS | 14 |
| Soti-15 | NFPVYDTTHHGGHRSKLH | 15 |
| Soti-16 | NVHPQSENTNTTRPHKSTQR | 16 |
| Soti-17 | QHGMHSPNLGARMNATPH | 17 |
| Soti-18 | RPNDTHHPGKCDTHAVCHQT | 18 |
| Soti-19 | SHLMHVKAPTDQASTRNRFD | 19 |
| Soti-20 | SSSTPPNSPKHSKYNVWTSP | 20 |
| Soti-21 | VHQTTPQHKDAVNLPRK | 21 |
| Soti-22 | WHSSEGQYKKPNNHRQYHTG | 22 |
| Soti-23 | YKHERHYSQPLKVRH | 23 |

### Example 2

### Characterization of Selected Peptides for Silica-Coated Titanium Dioxide (TiO₂) Particles Binding Activities

Enzyme-linked immunosorbent assay (ELISA) was used to evaluate the silica-coated particle binding affinity of the biopanning selected peptide candidates (Example 1; biotinylated peptides Phage ID: Soti-23, Soti-5, and Soti-8). The identified peptides were synthesized using a standard solid-phase synthesis method (U.S. patent application Ser. No. 11/251,715). All peptides were modified to contain a biotinylated lysine residue at the C-terminus of the amino acid binding sequence for detection purposes. See Tables 2 and 3.

Silica-coated TiO₂ particles and silica-coated iron oxide particles were separately dispersed in water at 2.5 mg/mL. The preparation of silica-coated particles is known in the art (see, for example, U.S. Pat. No. 2,885,366; hereby incorporated by reference). The dispersion was made by vortexing the mixture for 1 min, which resulted in an average particle size of approximately 0.5 μm in diameter. The particle dispersion (1 mL each) was then centrifuged for 2 min at 5000 rpm. The liquid portion was removed by aspirating it out of each tube. The tubes were incubated in SUPERBLOCK® blocking buffer (Pierce Chemical Company, Rockford, Ill.; Prod. #37535) for 1 hour at room temperature (approximately 22° C.), followed by 3 washes with TBST (TBS in 0.05% TWEEN® 20). Then tubes were then rinsed 3 times with wash buffer consisting of TBST-0.05% using the same centrifugation and aspiration steps. Peptide binding buffer consisting of 20 μM biotinylated peptides in TBST and 1 mg/mL BSA was added to the particles and incubated for 1 hour at room temperature (˜22° C.), followed by 6 TBST washes. Then, the streptavidin-alkaline phosphatase (AP) conjugate (Pierce Chemical Co., Rockford, Ill.) was added to each well at standard concentration and incubated for 1 hour at room temperature, followed by 6 times of washes with TBST. At the last wash. All particles were transferred to new tubes and then the color development and the absorbance measurements were performed. The resulting absorbance values, reported as the mean of at least three replicates, and the standard error of the mean (SEM) are given in Table 2 and Table 3.

The results demonstrate that all of three silica-binding peptides tested had a significant higher binding activity for silica coated particles than the control samples.

TABLE 2
Peptide Silica-Coated TiO₂ Particle Binding Results

| | | | | | Average | |
|---|---|---|---|---|---|---|
| | Amino Acid Sequence | Well | Well | Well | O.D. at | |
| Phage ID | (SEQ ID NO:) | 1 | 2 | 3 | 504 nm | SEM |
| Control | No peptide | 0.056 | 0.073 | 0.068 | 0.066 | 0.005 |
| Soti-23 | YKHERHYSQPLVKRH-K-Biotin | 1.481 | 1.216 | 1.471 | 1.389 | 0.087 |
| | (SEQ ID NO: 24) | | | | | |
| Soti-5 | DHNNRQHAVEVRENKTHTAR-K-Biotin | 1.415 | 1.044 | 1.28 | 1.246 | 0.108 |
| | (SEQ ID NO: 25) | | | | | |
| Soti-8 | HHDRAEPRGMAATLAQTI-K-Biotin | 0.562 | 0.8 | 0.746 | 0.703 | 0.071 |
| | (SEQ ID NO: 26) | | | | | |

TABLE 3
Peptide Silica-Coated Iron Oxide Particle Binding Results

| | | | | | Average | |
|---|---|---|---|---|---|---|
| | Amino Acid Sequence | Well | Well | Well | O.D. at | |
| Phage ID | (SEQ ID NO: ) | 1 | 2 | 3 | 504 nm | SEM |
| Control | No peptide | 0.043 | 0.049 | 0.038 | 0.043 | 0.003 |
| Soti-23 | YKHERHYSQPLKVRH-K-Biotin | 1.508 | 1.88 | 1.7 | 1.696 | 0.107 |
| | (SEQ ID NO: 24) | | | | | |
| Soti-5 | DHNNRQHAVEVRENKTHTAR-K-Biotin | 1.62 | 1.639 | 1.437 | 1.565 | 0.064 |
| | (SEQ ID NO: 25) | | | | | |
| Soti-8 | HHDRAEPRGMAATLAQTI-K-Biotin | 0.256 | 0.288 | 0.212 | 0.252 | 0.022 |
| | (SEQ ID NO: 26) | | | | | |

### Example 3

### Determination of the Binding Affinity of Silica-Binding Peptides

The purpose of this Example was to demonstrate the affinity of the silica-binding peptides for the silica-coated particle surface, measured as MB₅₀ values, using an ELISA assay.

Silica-binding peptides, Soti-23, Soti-5, and Soti-8 (see Tables 1 and 2) identified using the method described in Example 1 were synthesized by Synpep Inc. (Dublin, Calif.). Each peptide was biotinylated by adding biotin on to a C-terminal lysine residue added to the respective peptide.

### MB₅₀ Measurement of Silica-Binding Peptides:

The MB₅₀ measurements of biotinylated peptides binding to silica-coated TiO₂ were done using the 96-well plate format. Silica-coated particles were added to the wells. The wells containing the silica-coated TiO₂ were blocked with blocking buffer (SUPERBLOCK™ from Pierce Chemical Co., Rockford, Ill.) at room temperature (−22° C.) for 1 hour, followed by six washes with TBST-0.5%, 2 min each, at room temperature (˜22° C.). Various concentrations of biotinylated binding peptides were added to each well, incubated for 1 hour at room temperature (˜22° C.), and washed six times with TBST-0.5%, 2 min each, at room temperature (˜22° C.). Then, streptavidin-horseradish peroxidase (HRP) conjugate (Pierce Chemical Co., Rockford, Ill.) was added to each well (1.0 μg per well), and incubated for 1 hour at room temperature (˜22° C.). After the incubation, the wells were washed six times with TBST-0.5%, 2 min each at room temperature. Finally, the color development and the absorbance measurements were performed as described in Example 2.

The results were plotted as A₄₅₀ versus the concentration of peptide using GraphPad Prism 4.0 (GraphPad Software, Inc., San Diego, Calif.). The MB₅₀ values were calculated from Scatchard plots. The results are listed in Table 4.

TABLE 4
MB₅₀ Data for Various Silica-binding Peptides

| Peptide | Amino Acid Sequence | |
|---|---|---|
| ID | (SEQ ID NO: ) | MB₅₀ (M) |
| Soti-13 | LNSMSDKHHGHQNTATRNQH-K-Biotin | 2.9 × 10⁻⁹ M |
| | (SEQ ID NO: 27) | |
| Soti-23 | YKHERHYSQPLKVRH-K-Biotin | 9.0 × 10⁻⁹ M |
| | (SEQ ID NO: 24) | |
| Soti-8 | HHDRAEPRGMAATLAQTI-K-Biotin | 1.0 × 10⁻⁸ M |
| | (SEQ ID NO: 26) | |

### Example 4

### Peptide Performance for Uptake and Retention of Silica-Coated Iron Oxide Particles on Hair

The purpose of this example was to illustrate the performance of peptide-based reagents comprising at least one of the present silica-binding peptides on update and retention of silica-coated iron oxide particles on hair.

Several peptide-based reagents comprising at least one of the present silica-binding peptides were prepared using standard microbiological techniques. The design and sequence of the various peptide constructs is provided in Table 5.

TABLE 5
Sequences of Several Peptide-Based Reagents

| | | | |
|---|---|---|---|
| Peptide ID | Formula* | Peptide Sequence | SEQ ID NO: |

| | | | |
|---|---|---|---|
| HC316 | PG-Gray5-GGAGGAG-Gray5- | PGTAEIQSSKNPNPHPQRSWTNGGAGGAGTAEIQSS | 28 |
| | GGAGGAV-Soti5-GGAGGAG-Soti5- | KNPNPHPQRSWTNGGAGGAVDHNNRQHAVEVRENK | |
| | GK | THTARGGAGGAGDHNNRQHAVEVRENKTHTARGK | |
| HC317 | PG-Hair4-GGKGGAG-Hair4- | PGTPPELAHTPHHLAQTRLTDRGGKGGAGTPPELAHT | 29 |
| | GGAGGAV-Soti5-GGAGGAG-Soti5- | PHHLAQTRLTDRGGAGGAVDHNNRQHAVEVRENKTH | |
| | GK | TARGGAGGAGDHNNRQHAVEVRENKTHTARGK | |
| HC318 | PG-Hair5-GGAGGAG-Hair5- | PGHHGTHHNATKQKNHVGGAGGAGHHGTHHNATKQ | 30 |
| | GGAGGAV-Soti5-GGAGGAG-Soti5- | KNHVGGAGGAVDHNNRQHAVEVRENKTHTARGGAG | |
| | GK | GAGDHNNRQHAVEVRENKTHTARGK | |
| HC321 | PG-Hair4-GGKGGAG-Hair4- | PGTPPELAHTPHHLAQTRLTDRGGKGGAGTPPELAHT | 31 |
| | GGAGGAV-Soti8-GGAGGAG-Soti8- | PHHLAQTRLTDRGGAGGAVHHDRAEPRGMAATLAQTI | |
| | GK | GGAGGAGHHDRAEPRGMAATLAQTIGK | |
| HC325 | PG-Hair4-GGKGGAG-Hair4- | PGTPPELAHTPHHLAQTRLTDRGGKGGAGTPPELAHT | 32 |
| | GGAGGAV-Soti13-GGAGGAG-Soti13- | PHHLAQTRLTDRGGAGGAVLNSMSDKHHGHQNTATR | |
| | GK | NQHGGAGGAGLNSMSDKHHGHQNTATRNQHGK | |
| HC327 | PG-Gray3-GGAGGAG-Gray3- | PGHDHKNQKETHQRHAAGGAGGAGHDHKNQKETHQ | 33 |
| | GGAGGAV-Soti23-GGAGGAG-Soti23- | RHAAGGAGGAVYKHERHYSQPLKVRHGGAGGAGYK | |
| | GK | HERHYSQPLKVRHGK | |
| HC330 | PG-Hair5-GGAGGAG-Hair5- | PGHHGTHHNATKQKNHVGGAGGAGHHGTHHNATKQ | 34 |
| | GGAGGAV-Soti23-GGAGGAG-Soti23- | KNHVGGAGGAVYKHERHYSQPLKVRHGGAGGAGYK | |
| | GK | HERHYSQPLKVRHGK | |

*= binding peptides in bold. Peptide linkers are italicized. Peptide bridges are underlined.

### Small Hair Tress.

A 2-3 mm wide strip of a polyurethane-based adhesive (e.g. 3M SCOTCH-GRIP™ 4475 Plastic Adhesive) was placed on a TEFLON® sheet. Hair to be tufted was spread out to 2-3 mm thickness and placed over the adhesive glue. Another 1-2 mm wide strip of adhesive was placed on the top side and the glue-line was pressed down using a TEFLON®-covered metal bar to a thickness of 1-1.5 mm. The adhesive was dried for 6-12 hours. Hair samples (natural white; International Hair Importers and Products (Bellerose, N.Y.)) were peeled off and cut 1.5 to 2.0 cm away from the glue-line. The hair swatches were cut to 5-6 mm width to yield tufts of 60-80 mg hair.

### Pigment Dispersion.

Red iron oxide pigment particles (Unipure Red LC381; Sensient Technologies Corp, Milwaukee, Wis.) were coated with an effective amount of silica. Approximately 500 mg of the silica-coated iron oxide pigment (average particle size of 200-300 nm (d50)), 5-mL of buffer (25 mM tris HCl, pH 7.5), and 15 g of 0.7 mm zirconia beads (BioSpec #1107907zx) were mixed in FlackTek SPEEDMIXER™ (FlackTek Inc., Landrum, S.C.) at 3000 rpm for 5 min. After cooling for 5 min the contents were mixed again for an additional 5 min. Supernatant (dispersion) was separated from the beads by suction. The final concentration of the pigment in the aqueous dispersion was approximately 10 wt %.

Pretreatment with Peptide.

Peptide (0.5 mg) was dissolved in 0.5 mL of buffer (25 mM tris.HCl, pH 7.5). A small tress of natural white hair (International Hair Importers and Products) was suspended in the peptide solution in a vial and agitated at a low speed on a vortex mixer for 30 minutes. The tress was rinsed with the treatment-buffer twice followed by a thorough rinse under a jet of de-ionized water.

### Pigment Application.

The peptide-pretreated hair tress was treated with a 0.4% pigment dispersion in 25 mM tris.HCl (made by dilution of 20 μL of 10% dispersion in 0.5 mL buffer) in a vial at slow agitation. After 30 minutes, the tress was thoroughly rinsed under a jet of de-ionized water and dried in air. L*, a* and b* values for color uptake were measured using a spectrophotometer.

### Shampoo Cycle.

The tresses subjected to shampoo cycle were placed in wells of a 24-well plate. Glass and stainless steel beads (3 mm glass beads ×4, 4 mm stain steel beads ×1, 6.35 mm glass beads ×2) were charged into each well. Approximately 1.0 mL of 0.2% sodium lauryl ether sulfate (SLES) solution was added to each well. The well plate was covered with a flexible SANTOPRENE® mat and was agitated at high speed on the vortex mixer for 30 sec. The shampoo was removed from the wells by suction. Approximately 4 mL of de-ionized water was added to each well; the plate was agitated at a low speed on the vortex mixer for 5-10 sec. The rinse solution was removed by suction. The tress was thoroughly rinsed under a jet of de-ionized water and subjected to the next shampoo cycle. After the last shampoo cycle, the tress was dried in air and the retained color is measured.

Delta-E values are calculated from L*, a* and b* using the formula

$$Δ E uptake = \sqrt{( {( {Lu}^{*} - L 0 )}^{⋀} 2 + {( {au}^{*} - a 0 )}^{⋀} 2 + {( {bu}^{*} - b 0 )}^{⋀} 2 )}$$

$$and$$

$$Δ E retention = \sqrt{( {( {Lr}^{*} - L 0 )}^{⋀} 2 + {( {ar}^{*} - a 0 )}^{⋀} 2 + {( {br}^{*} - b 0 )}^{⋀} 2 )}$$

### Where,

Lu*, au* and bu* are L*, a* and b* values for a sample tress after color uptake,
Lr*, ar* and br* are L*, a* and b* values for a sample tress after shampoo cycles, and
L0*, a0* and b0* are L*, a* and b* values for untreated natural white hair.

TABLE 6
Uptake and Retention of Silica-Coated Particles on Hair.

| | | | | ΔE |
|---|---|---|---|---|
| | | | | retention - |
| Peptide | Peptide | Pigment | ΔE | 2 shampoo |
| ID | amount, mg | (mg) | uptake | cycles |
| HC316 | 0.5 | 2 | 11 | 7 |
| HC317 | 0.5 | 2 | 17 | 4 |
| HC318 | 0.5 | 2 | 21 | 6 |
| HC321 | 0.5 | 2 | 12 | 9 |
| HC325 | 0.5 | 2 | 28 | 4 |
| HC327 | 0.5 | 2 | 30 | 16 |
| HC330 | 0.5 | 2 | 26 | 10 |

### Example 5

### Selection of Tooth (Pellicle) Binding Peptides Using Standard Biopanning

The purpose of this Example was to identify phage peptides that bind tooth pellicle formed in vivo on bovine enamel using phage display biopanning.

Bovine enamel incisors were obtained from SE Dental (Baton Rouge, La.). The teeth were cut to approximately 5 mm squares and polished to remove surface debris. Enamel blocks were sterilized and sewn into intra-oral retainers in order to expose the enamel surface to the human oral environment. A retainer with 2 to 4 enamel blocks was worn in the human mouth for 30 min to form a pellicle layer on the enamel. After incubation, the enamel blocks were removed from the retainer, rinsed with water and embedded in a well plate contained molding material so as to only expose the pellicle coated enamel surface in the well. The plate was sterilized with UV light for 10 minutes.

The substrates were then incubated in blocking buffer for 1 hour at room temperature (1 mg/mL Bovine Serum Albumin in Phosphate Buffered Saline pH 7.2 (Pierce BUPH™ #28372) with 0.1% TWEEN® 20 (PBST), followed by 2 washes with PBST. Libraries of phage containing random peptide inserts (10″ pfu) from 15 to 20 amino acids were added to each well. After 30 minutes of incubation at 37° C. and shaking at 50 rpm, unbound phage were removed by aspirating the liquid out of each well followed by 6 washes with 1.0 mL PBST.

The enamel blocks were then transferred to clean tube and 1 mL of elution buffer consisting of 1 mg/mL BSA in 0.2 M glycine-HCl, pH 2.2, was added to each well and incubated for 10 min to elute the bound phages. Then, 167 μL of neutralization buffer consisting of 1 M Tris-HCl, pH 9.1, was added to each well. The phage particles, which were in the elution buffer as well as on the enamel blocks, were amplified by incubating with 20 mL diluted E. coli ER2738 cells, from an overnight culture diluted 1:100 in LB medium, at 37° C. for 4.5 h. After this time, the cell culture was centrifuged for 2 min and the upper 15 mL of the supernatant was transferred to a fresh tube, 2.5 mL of PEG/NaCl (20% polyethylene glycol-800, 2.5 M sodium chloride) was added, and the phage was allowed to precipitate overnight at 4° C. The precipitate was collected by centrifugation at 10,000×g at 4° C. and the resulting pellet was resuspended in 1 mL of PBS. This was the first round of amplified stock. The amplified first round phage stock was then tittered according to the standard protocol. For subsequent rounds of biopanning, more than 2×10¹¹ pfu of phage stock from the previous round was used. Each additional round after the first also included additional washes with 0.5% sodium lauryl sulfate in water (Spectrum), two washes with carbonate buffer pH 9.4 (Pierce BUPH™ Carbonate-Bicarbonate Buffer #28382) and 2 washes with 50 mM phosphate buffer, pH 2.5.

The biopanning process was repeated an additional 3 more rounds under the same conditions as described above with an additional exposure of the phage stock to oral soft tissue. The phage stock amplified from the 2^{rd} round was exposed first to EPIORAL™ and EPIGINGIVAL™ soft tissues (MatTek Corp, Ashland, Mass.) by incubating 8 μL of the 2^{nd} round phage stock+42 μL of blocking buffer (PBST+1 mg/mL BSA) for 60 min. The solution was removed from the tissue and an additional 50 μL of PBS was incubated with the tissue for 30 min. The solutions were combined and used in additional rounds of biopanning as described above.

After the 3rd round of biopanning and each subsequent round, 95 random single phage plaques were isolated and the single stranded phage genomic DNA was prepared using the illustra Templiphi 500 Amplification Kit (GE Healthcare, Piscataway, N.J.) and sequenced at the DuPont Sequencing Facility using −96 gill sequencing primer (5′-CCCTCATAGTTAGCGTAACG-3′; SEQ ID NO: 262). The displayed peptide is located immediately after the signal peptide of gene III. Based on the peptide sequences, 31 phage candidates were identified for further pellicle binding analysis.

TABLE 7
Toothbinding Peptides Identified from Biopanning
on 30 mm in vivo Pellicle

| Peptide | | |
|---|---|---|
| ID | Amino Acid Sequence | SEQ ID NO |
| DenP 01 | NGNNHTDIPNRSSYTGGSFA | 263 |
| DenP 02 | TMTNHVYNSYTEKHSSTHRS | 264 |
| DenP 03 | TTYHYKNIYQESYQQRNPAV | 265 |
| DenP 04 | VEPATKNMREARSSTQMRRI | 266 |
| DenP 05 | YLLPKDQTTAPQVTPIVQHK | 267 |
| DenP 06 | ASNLDSTFTAINTPACCT | 268 |
| DenP 07 | EFPYYNDNPPNPERHTLR | 269 |
| DenP 08 | GMPTRYYHNTPPHLTPKF | 270 |
| DenP 09 | HKNAIQPVNDATTLDTTM | 271 |
| DenP 10 | AVVPADLNDHANHLS | 272 |
| DenP 11 | DLGTFPNRTLKMAAH | 273 |
| DenP 12 | FDGIGLGTATRHQNR | 274 |
| DenP 13 | QAAQVHMMQHSRPTT | 275 |
| DenP 14 | SEARARTFNDHTTPMPII | 276 |
| DenP 15 | ELDHDSRHYMNGLQRKVT | 277 |
| DenP 16 | GPQHVLMQDTHQGYAFDN | 278 |
| DenP 17 | TTGSSSQADTSASMSIVPAH | 279 |
| DenP 18 | KAPIANMLQPHSYQYSVA | 280 |
| DenP 19 | TYQGVPSWPAVIDDAIRR | 281 |
| DenP 20 | VNPNWVETQALHQPPGNT | 282 |
| DenP 21 | DHNNRQHAVEVRENKTHTAR | 283 |
| DenP 22 | IYPNESMSTSNVRGPYHP | 284 |
| DenP 23 | HDPNHLTHQARTIYRNANHT | 285 |
| DenP 24 | SNATMYNIQSHSHHQ | 286 |
| DenP 25 | ANELSTYAQTNPGSG | 287 |
| DenP 26 | DTIHPNKMKSPSSPL | 288 |
| DenP 28 | APPTYQTASYPHNLPSKRKM | 289 |
| DenP 29 | QVPDYLSPTHQKKAFLEIPT | 290 |
| DenP 30 | TNDLHANPFTGTYIAPDPTS | 291 |
| DenP 32 | HKNENIMQYNVNDRWHITPA | 292 |
| DenP 33 | IDGPHHSPVHRYHTPSIT | 293 |

### Example 6

### Characterization of Tooth (Pellicle) Binding Candidates on Pellicle Surface

A total of 29 selected phage candidates from Table 7 were used in phage ELISA Experiment to determine binding affinity and coverage of each phage on pellicle substrates. Purified phage lysates were used for binding to pellicle coated bovine enamel using an anti-M13 phage antibody conjugated to horseradish-peroxidase, followed by the addition of chromogenic agent TMB, obtained from Pierce Biotechnology (Rockford, Ill.). The plates were read at A₄₅₀ₙₘ.

Enamel substrates were cut to approximately 7 mm squares and mounted on wax mounting for incubation in the mouth for 30 min to form a pellicle coated surface. The pellicle coated enamel substrates were removed from the wax backing and placed in well plates with the pellicle surface exposed as in Example 5. Each pellicle coated substrate was incubated for 1.5 h at room temperature with 1 mL of blocking buffer, consisting of 1 mg/mL BSA in PBST (Pierce BUPH™ #28372 with 0.1% TWEEN® 20). The blocking buffer was removed by aspirating the liquid out of each well. The tube was rinsed 2 times with wash buffer consisting of PBST. The wells were filled with 1 mL of 10″ pfu purified phage stock which was prepared by diluting in blocking buffer. The samples were incubated at room temperature for 30 min with slow shaking at 37° C. The non-binding phage was removed by washing 5 times with PBST. Then, 500 μL of horseradish peroxidase/anti-M13 antibody conjugate (Amersham USA, Piscataway, N.J.), diluted 1:500 in the blocking buffer, was added and incubated for 1 h at room temperature. The conjugate solution was removed and was washed 3 times with PBST. Each enamel substrate was removed from the well and washed again in a 15-mL test tube with 5 mL of PBST. Each enamel substrate was then mounted in a clean well plate with only the enamel surface exposed. A 1:1 solution of TMB substrate and H₂O₂ (200 μL), obtained from Pierce Biotechnology (Rockford, Ill.) was added to each well and the color was allowed to develop for between 5 to 30 min, typically for 10 min, at room temperature (approximately 22° C.). Then, stop solution (100 μL of 2 M H₂SO₄) was added to each well and the solution was transferred to a 96-well plate and the A₄₅₀ was measured using a microplate spectrophotometer (Molecular Devices, Sunnyvale, Calif.). The resulting absorbance values, are given in Table 8. The analysis of all 30 pellicle binding candidates was completed over the course of two days and the results were normalized to an internal control.

TABLE 8
Phage ELISA Results for Pellicle-binding Peptide
Candidates Obtained from Biopanning

| | | | O.D. at |
|---|---|---|---|
| Peptide | | SEQ | 450 nm |
| ID | Amino Acid Sequences | ID NO: | (normalized) |
| Control | IPWWNIRAPLNAGGG | 294 | 1.000 |
| DenP 01 | NGNNHTDIPNRSSYTGGSFA | 263 | 1.002 |
| DenP 02 | TMTNHVYNSYTEKHSSTHRS | 264 | 1.951 |
| DenP 03 | TTYHYKNIYQESYQQRNPAV | 265 | 2.495 |
| DenP 04 | VEPATKNMREARSSTQMRRI | 266 | 1.421 |
| DenP 05 | YLLPKDQTTAPQVTPIVQHK | 267 | 1.087 |
| DenP 07 | EFPYYNDNPPNPERHTLR | 269 | 1.500 |
| DenP 08 | GMPTRYYHNTPPHLTPKF | 270 | 1.182 |
| DenP 09 | HKNAIQPVNDATTLDTTM | 271 | 1.364 |
| DenP 10 | AVVPADLNDHANHLS | 272 | 1.619 |
| DenP 11 | DLGTFPNRTLKMAAH | 273 | 1.663 |
| DenP 12 | FDGIGLGTATRHQNR | 274 | 2.079 |
| DenP 13 | QAAQVHMMQHSRPTT | 275 | 0.845 |
| DenP 14 | SEARARTFNDHTTPMPII | 276 | 2.498 |
| DenP 15 | ELDHDSRHYMNGLQRKVT | 277 | 1.112 |
| DenP 16 | GPQHVLMQDTHQGYAFDN | 278 | 2.190 |
| DenP 17 | TTGSSSQADTSASMSIVPAH | 279 | 0.971 |
| DenP 18 | KAPIANMLQPHSYQYSVA | 280 | 1.143 |
| DenP 19 | TYQGVPSWPAVIDDAIRR | 281 | 1.052 |
| DenP 20 | VNPNWVETQALHQPPGNT | 282 | 1.298 |
| DenP 21 | DHNNRQHAVEVRENKTHTAR | 283 | 0.728 |
| DenP 22 | IYPNESMSTSNVRGPYHP | 284 | 1.420 |
| DenP 23 | HDPNHLTHQARTIYRNANHT | 285 | 1.236 |
| DenP 24 | SNATMYNIQSHSHHQ | 286 | 0.979 |
| DenP 25 | ANELSTYAQTNPGSG | 287 | 0.909 |
| DenP 26 | DTIHPNKMKSPSSPL | 288 | 1.039 |
| DenP 28 | APPTYQTASYPHNLPSKRKM | 289 | 1.203 |
| DenP 29 | QVPDYLSPTHQKKAFLEIPT | 290 | 0.976 |
| DenP 30 | TNDLHANPFTGTYIAPDPTS | 291 | 1.082 |
| DenP 32 | HKNENIMQYNVNDRWHITPA | 292 | 1.441 |

### Example 7

### Peptide Performance for Uptake and Retention of Silica-Coated Iron Oxide Particles on Tooth Pellicle Surfaces

The purpose of this example was to illustrate the performance of peptide-based reagents comprising at least one of the present silica-binding peptides on uptake and retention of silica-coated particles on tooth surfaces.

Several peptide-based reagents comprising at least one of the present silica-binding peptides were prepared using standard microbiological techniques and chemical techniques. Additional peptides not incorporating silica-binding sequences were also produced. The design and sequence of the various peptide constructs is provided in Table 9. Each of these peptides incorporate DenP03 sequence discovered by panning on pellicle surfaces. Confirmation of binding of these peptides on pellicle surfaces were completed using an ELISA assay. All peptides in Table 9 were found to bind equivalently to the pellicle surface.

TABLE 9
Sequences of Several Peptide-Based Reagents

| | | | SEQ |
|---|---|---|---|
| Peptide ID | Formula* | Peptide Sequence | ID NO: |
| DenP03-H6 | PS-SSRP-DenP03- | PSSSRPTTYHYKNIYQ | 295 |
| | HHHHHH | ESYQQRNPAVHHHHHH | |
| DE99 | PS-SSRP-DenP03- | PSSSRPTTYHYKNIYQ | 296 |
| | GP-TonB-PAGP- | ESYQQRNPAVGPEPEP | |
| | HHHHHH | EPEPIPEPPKEAPVVI | |
| | | EKPKPKPKPKPKPPAG | |
| | | PHHHHHH | |
| DE117 | PS-SSRP-DenP03- | PSSSRPTTYHYKNIYQ | 297 |
| | GP-TonB-PA-SSRP- | ESYQQRNPAVGPEPEP | |
| | DenP03-GP- | EPEPIPEPPKEAPVVI | |
| | HHHHHH | EKPKPKPKPKPKPPAS | |
| | | SRPTTYHYKNIYQESY | |
| | | QQRNPAVGPHHHHHH | |
| DE62 | P-SSRP-DenP03-GP- | PSSRPTTYHYKNIYQE | 298 |
| | TonB-PA-SSRP- | SYQQRNPAVGPEPEPE | |
| | DenP03- | PEPIPEPPKEAPVVIE | |
| | GSSGPGSP-Soti23- | KPKPKPKPKPKPPASS | |
| | GSG-Soti23-GP- | RPTTYHYKNIYQESYQ | |
| | HHHHHH | QRNPAVGSSGPGSPYK | |
| | | HERHYSQPLKVRHGSG | |
| | | YKHERHYSQPLKVRHG | |
| | | PHHHHHH | |

*= binding peptides in bold. Peptide linkers are italicized. Peptide bridges are underlined.

Bovine enamel incisors were obtained from SE Dental (Baton Rouge, La.). Teeth were sectioned and cut into enamel slabs approximately 7 mm on each side using a DREMEL® rotary saw (Robert Bosch Power Tool Corporation; Chicago, Ill.) with a diamond blade. The enamel slabs were cleaned and lightly polished to remove surface debris. Each enamel block was mounted on wax mounting and sterilized with ethylene oxide. Mounted enamel bocks were incubated in the mouth for 30 min to form a pellicle coated surface. The pellicle-coated enamel substrates were removed from the wax backing, rinsed with water and placed in well plates. Each pellicle coated enamel slab was measured for color using a Konica-Minolta 2600d integrating sphere spectrophotometer.

Red iron oxide pigment particles (Unipure Red LC381; Sensient Technologies Corp, Milwaukee, Wis.) was first coated with silica by a process described in U.S. Pat. No. 2,885,366 to Iler (incorporated herein by reference) with a 3.8% silica loading. The coated particles were dispersed by mixing 10 g of dry pigment, 25 g of mm zirconia beads (BioSpec Products, Inc., Bartlesville, Okla.; catalog #11079105z) and 73 g water in a FlackTek SPEEDMIXER™ (FlackTek Inc., Landrum, S.C.) at 3000 rpm for 5 min. The sample was put on ice for 10 min and then mixed again at 3000 prm for 5 min. This was repeated 2 additional times. Supernatant (dispersion) was separated from the beads by filtration. The final concentration of the pigment in the aqueous dispersion was approximately 12 wt %. The dispersion average particle size was approximately 250 nm as measured by dynamic light scattering. For application to pellicle surfaces, a working concentration of 0.5 wt % was made by diluting in 10 mM sodium phosphate buffer at pH 7.2.

Peptide was first dissolved in molecular grade water to a concentration of 200 μM. Stock solutions for application to pellicle surface were made by diluting to a final concentration of 20 μM in phosphate buffered saline at pH 7.2 (Pierce).

Each pellicle coated enamel block was exposed to either 1 mL PBS buffer (no peptide control) or 1 mL of 20 μM peptide as listed in Table 10 for 1 min. Upon removal from the peptide solution, the enamel block was dipped in water to rinse and then added to 1 mL of 0.5% silica-coated red iron oxide dispersion for 5 min. The enamel block was dipped in water several times to rinse off unbound pigment. Color was measured again for each enamel block to obtain L*, a* and b* values and compare to initial color to measure color uptake.

Delta-E values are calculated from L*, a* and b* using the formula

$$Δ E uptake = \sqrt{( {( Lu ⋆ - L 0 )}^{⋀} 2 + {( {au}^{*} - a 0 )}^{⋀} 2 + {( {bu}^{*} - b 0 )}^{⋀} 2 )}$$

### Where,

Lu*, au* and bu* are L*, a* and b* values for a enamel block following pigment application.,
L0*, a0* and b0* are L*, a* and b* values for initial color for each enamel block

TABLE 10
Deposition of Silica-Coated Particles on Pellicle-coated Enamel

| | Peptide | Pigment | Silica | |
|---|---|---|---|---|
| Peptide | concentration | concentration | binding | ΔE |
| ID | (uM) | (% solids) | peptide | uptake |
| none | 0 | 0.5 | n/a | 6.5 |
| DenP03-H6 | 20 | 0.5 | — | 10.1 |
| DE99 | 20 | 0.5 | — | 22.3 |
| DE117 | 20 | 0.5 | — | 24.2 |
| DE62 | 20 | 0.5 | Soti-23 | 43.7 |

The results confirm that enhanced binding of silica-coated red iron oxide particles to pellicle was achieved with peptides incorporating a silica-binding sequence discovered with panning.

### Example 8

### Tooth Whitening Using Silica-Coated Titanium Dioxide

The purpose of this example was to confirm the performance of peptide-based reagents that demonstrate uptake of silica-coated red iron oxide particles also function to uptake silica-coated titanium dioxide for use as tooth whitening compositions.

Silica-coated rutile titanium dioxide (Luce WP-10S) was obtained from U.S. Cosmetics Corp. (Dayville, Conn.). A 12 wt % solution of the pigment was prepared with Millipore water. The solution was dispersed by horn sonication using a Branson SONIFIER® 150 ultrasonic cell disruptor (Branson Instruments, Inc., Danbury, Conn.) at 10 W. The solution was sonicated in an ice bath for 6 min total, with 2 min sonication intervals. A working solution of 0.5 wt % in 10 mM sodium phosphate buffer at pH 7.2 was made for application to peptide-coated enamel.

Bovine enamel incisors were obtained from SE Dental (Baton Rouge, La.). Teeth were sectioned and cut into enamel slabs approximately 7 mm on each side using a DREMEL® rotary saw (Robert Bosch Power Tool Corporation; Chicago, Ill.) with a diamond blade. The enamel slabs were cleaned and lightly polished to remove surface debris. The enamel was pretreated with a mixture of coffee and tea in order to stain to a color similar to human stained teeth. Each enamel block was then mounted on wax mounting and sterilized with ethylene oxide. Mounted enamel bocks were incubated in the mouth for 30 min to form a pellicle coated surface. The pellicle coated enamel substrates were removed from the wax backing, rinsed with water and placed in well plates. Each pellicle coated enamel slab was measured for color using a Konica-Minolta 2600d integrating sphere spectrophotometer (Konica Minolta Holdings, Inc., Tokyo, Japan)

Peptide DE62 (SEQ ID NO: 298) stock solution was prepared as described in Example 7. A 10 μM working concentration was made in PBS. Each enamel block was treated with buffer or DE62 peptide for 30 min. The enamel blocks were rinsed and exposed to the silica-coated titanium dioxide dispersion for 30 min. Color was measured again for each enamel block to obtain L*, a* and b* values and whiteness index to compare to initial color values. Enamel blocks first with DE62 peptide bound to the surface produced a visually perceptible whitening effect on the stained enamel. Table 11 provides the before and after color comparison. Whiteness index (WI) is defined by the International Commission on Illumination (CIE) and described in ASTM method E313-05 and calculated for D65/10 incident light as:

WI=Y+800*(0.3138−x)+1700*(0.3310−y)

Where Y, x, and y are the luminance factor and the chromaticity coordinates respectively of the enamel substrate.

TABLE 11
Whitening Performance of Peptide-mediated Deposition of
Silica-coated Titanium Dioxide on Stained Enamel.

| | | L* | a* | b* | WI | ΔWI |
|---|---|---|---|---|---|---|
| No | Before | 66.1 | 6.0 | 16.3 | −62.0 | 1.4 |
| peptide | After | 62.5 | 6.4 | 14.7 | −60.7 | |
| DE62 | Before | 67.8 | 4.1 | 10.1 | −21.8 | 32.4 |
| (SEQ ID | After | 70.3 | 2.7 | 5.3 | 10.6 | |
| NO: 298) | | | | | | |

The increase in whiteness index and decrease in b* indicate the color of the enamel block has been modified with the addition of the peptide-silica-coated titanium dioxide complex to provide a visible whitening effect on the enamel surface.

What is claimed is:

1. A silica-binding peptide having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, and 27.

2. A peptide-based reagent selected from the group consisting of:
a) a peptide-based reagent having the general structure:
[(BSBP)ₘ-(SiBP)ₙ]ₓ; and
b) a peptide-based reagent having the general structure:
[[(BSBP)ₘ-S_{q}]ₓ-[(siBP)ₙ-Sᵣ]_{z}]_{y},;
wherein
a) BSBP is a body surface-binding peptide;
b) SiBP is a silica-binding peptide having an amino acid sequence selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, and 27;
c) S is a spacer;
d) m, n, x and z independently range from 1 to about 10;
e) y is from 1 to 5; and
f) q an r are each independently 0 or 1, provided that both r and q may not be 0.

3. The peptide-based reagent according to claim 2 wherein the body surface-binding peptide is from about 7 to about 60 amino acids.

4. The peptide-based reagent according to claim 2 wherein the spacer is a peptide linker or a peptide bridge comprising a length of 1 amino acid to 60 amino acids.

5. The peptide-based reagent according to claim 3 wherein the body surface-binding peptide binds to a body surface selected from the group consisting of hair, skin, nail, and tooth.

6. The peptide-based reagent according to claim 2 wherein the silica-binding peptide has affinity for at least one silica-coated pigment.

7. The peptide-based reagent of claim 2 wherein the spacer is selected from the group consisting of ethanolamine, ethylene glycol, polyethylene with a chain length of 6 carbon atoms, polyethylene glycol with 3 to 6 repeating units, phenoxyethanol, propanolamide, butylene glycol, butylene glycolamide, propyl phenyl chains, ethyl alkyl chains, propyl alkyl chains, hexyl alkyl chains, steryl alkyl chains, cetyl alkyl chains, and palmitoyl alkyl chains.

8. The peptide-based reagent according to claim 2 wherein the peptide-based reagent is from about 14 amino acids to about 600 amino acids in length.

9. A personal care composition comprising the peptide-based reagent of claim 2 and at least one silica-coated particulate benefit agent.

10. The personal care composition of claim 9 wherein the at least one silica-coated particulate benefit agent is at least one silica-coated colorant.

11. The personal care composition of claim 10 wherein said personal care composition is an oral care composition selected from the group consisting of a toothpaste, a dental cream, a gel or tooth powder, a mouth wash, a breath freshener, and a dental floss.

12. A method for coupling a silica-coated particulate benefit agent to a body surface comprising:
a) providing:
i) at least one silica-coated particulate benefit agent; and
ii) at least one of the peptide-based reagents of claim 2; and
b) applying the at least one silica-coated particulate benefit agent of (a)(i) and the at least one peptide-based reagents of (a)(ii) to a body surface whereby the peptide-based reagent couples the silica-coated particulate benefit agent to the body surface.

13. The method according to claim 12 wherein the silica-coated benefit agent is a silica-coated colorant.

14. The method according to claim 12 wherein the body surface is selected from the group consisting of hair, skin, nail, and tooth.

15. The method according to claim 12 further comprising
c) applying at least one polymeric sealant to the body surface subsequent to step (b).

16. A method according to claim 15 wherein the at least one polymeric sealant is selected from the group consisting of poly(allylamine), acrylates, acrylate copolymers, polyurethanes, carbomers, methicones, amodimethicones, polyethylenene glycol, beeswax, siloxanes, linear polyvinylpyrrolidone homopolymers, cross-linked polyvinylpyrrolidone homopolymers, and combinations thereof.

17. The method according to claim 13 wherein the body surface is tooth.

18. The method according to claim 17 where the silica-coated colorant is a silica-coated white pigment.


SEQUENCE LISTING (298 sequences)

SEQ ID NO: 1 - protein, 20 aa, artificial sequence
  Silica-binding peptide
AEAKRHPVVPLHEQHGHHEL

SEQ ID NO: 2 - protein, 18 aa, artificial sequence
  Silica-binding peptide
APQTWNRPHPGHPNVHTR

SEQ ID NO: 3 - protein, 20 aa, artificial sequence
  Silica-binding peptide
ATTPPSGKAAAHSAARQKGN

SEQ ID NO: 4 - protein, 20 aa, artificial sequence
  Silica-binding peptide
DGRPDNPKHQQSYNRQLPRQ

SEQ ID NO: 5 - protein, 20 aa, artificial sequence
  Silica-binding peptide
DHNNRQHAVEVRENKTHTAR

SEQ ID NO: 6 - protein, 20 aa, artificial sequence
  Silica-binding peptide
GPEPRALNPKRHMDPATQIR

SEQ ID NO: 7 - protein, 15 aa, artificial sequence
  Silica-binding peptide
HDHHQTHNVLHGMKK

SEQ ID NO: 8 - protein, 18 aa, artificial sequence
  Silica-binding peptide
HHDRAEPRGMAATLAQTI

SEQ ID NO: 9 - protein, 20 aa, artificial sequence
  Silica-binding peptide
HHNHMTGADNPIFHNNTAHR

SEQ ID NO: 10 - protein, 18 aa, artificial sequence
  Silica-binding peptide
HNHAQMLRPEPTGISHKN

SEQ ID NO: 11 - protein, 20 aa, artificial sequence
  Silica-binding peptide
HTNDNGQSTPRRDPPAFQRK

SEQ ID NO: 12 - protein, 18 aa, artificial sequence
  Silica-binding peptide
HTNHHYDQKMHGPLPTPY

SEQ ID NO: 13 - protein, 20 aa, artificial sequence
  Silica-binding peptide
LNSMSDKHHGHQNTATRNQH

SEQ ID NO: 14 - protein, 20 aa, artificial sequence
  Silica-binding peptide
MHKPNNPDTHRSTPSPLGKS

SEQ ID NO: 15 - protein, 18 aa, artificial sequence
  Silica-binding peptide
NFPVYDTTHHGGHRSKLH

SEQ ID NO: 16 - protein, 20 aa, artificial sequence
  Silica-binding peptide
NVHPQSENTNTTRPHKSTQR

SEQ ID NO: 17 - protein, 18 aa, artificial sequence
  Silica-binding peptide
QHGMHSPNLGARMNATPH

SEQ ID NO: 18 - protein, 20 aa, artificial sequence
  Silica-binding peptide
RPNDTHHPGKCDTHAVCHQT

SEQ ID NO: 19 - protein, 20 aa, artificial sequence
  Silica-binding peptide
SHLMHVKAPTDQASTRNRFD

SEQ ID NO: 20 - protein, 20 aa, artificial sequence
  Silica-binding peptide
SSSTPPNSPKHSKYNVWTSP

SEQ ID NO: 21 - protein, 17 aa, artificial sequence
  Silica-binding peptide
VHQTTPQHKDAVNLPRK

SEQ ID NO: 22 - protein, 20 aa, artificial sequence
  Silica-binding peptide
WHSSEGQYKKPNNHRQYHTG

SEQ ID NO: 23 - protein, 15 aa, artificial sequence
  Silica-binding peptide
YKHERHYSQPLKVRH

SEQ ID NO: 24 - protein, 16 aa, artificial sequence
  Silica-binding peptide
YKHERHYSQPLKVRHK

SEQ ID NO: 25 - protein, 21 aa, artificial sequence
  Silica-binding peptide
DHNNRQHAVEVRENKTHTARK

SEQ ID NO: 26 - protein, 19 aa, artificial sequence
  Silica-binding peptide
HHDRAEPRGMAATLAQTIK

SEQ ID NO: 27 - protein, 21 aa, artificial sequence
  Silica-binding peptide
LNSMSDKHHGHQNTATRNQHK

SEQ ID NO: 28 - protein, 105 aa, artificial sequence
  synthetic construct
PGTAEIQSSKNPNPHPQRSWTNGGAGGAGTAEIQSSKNPNPHPQRSWTNGGAGGAVDHNNRQHAVEVRENKTHTARGGAGGAGDHNNRQHAVEVRENKTHTARGK

SEQ ID NO: 29 - protein, 105 aa, artificial sequence
  synthetic construct
PGTPPELAHTPHHLAQTRLTDRGGKGGAGTPPELAHTPHHLAQTRLTDRGGAGGAVDHNNRQHAVEVRENKTHTARGGAGGAGDHNNRQHAVEVRENKTHTARGK

SEQ ID NO: 30 - protein, 95 aa, artificial sequence
  synthetic construct
PGHHGTHHNATKQKNHVGGAGGAGHHGTHHNATKQKNHVGGAGGAVDHNNRQHAVEVRENKTHTARGGAGGAGDHNNRQHAVEVRENKTHTARGK

SEQ ID NO: 31 - protein, 101 aa, artificial sequence
  synthetic construct
PGTPPELAHTPHHLAQTRLTDRGGKGGAGTPPELAHTPHHLAQTRLTDRGGAGGAVHHDRAEPRGMAATLAQTIGGAGGAGHHDRAEPRGMAATLAQTIGK

SEQ ID NO: 32 - protein, 106 aa, artificial sequence
  synthetic construct
DPGTPPELAHTPHHLAQTRLTDRGGKGGAGTPPELAHTPHHLAQTRLTDRGGAGGAVLNSMSDKHHGHQNTATRNQHGGAGGAGLNSMSDKHHGHQNTATRNQHGK

SEQ ID NO: 33 - protein, 86 aa, artificial sequence
  synthetic construct
DPGHDHKNQKETHQRHAAGGAGGAGHDHKNQKETHQRHAAGGAGGAVYKHERHYSQPLKVRHGGAGGAGYKHERHYSQPLKVRHGK

SEQ ID NO: 34 - protein, 85 aa, artificial sequence
  synthetic construct
PGHHGTHHNATKQKNHVGGAGGAGHHGTHHNATKQKNHVGGAGGAVYKHERHYSQPLKVRHGGAGGAGYKHERHYSQPLKVRHGK

SEQ ID NO: 35 - protein, 12 aa, artificial sequence
  synthetic hair-binding peptide
RVPNKTVTVDGA

SEQ ID NO: 36 - protein, 12 aa, artificial sequence
  Synthetic construct
DRHKSKYSSTKS

SEQ ID NO: 37 - protein, 12 aa, artificial sequence
  Synthetic construct
KNFPQQKEFPLS

SEQ ID NO: 38 - protein, 12 aa, artificial sequence
  Synthetic construct
QRNSPPAMSRRD

SEQ ID NO: 39 - protein, 12 aa, artificial sequence
  Synthetic construct
TRKPNMPHGQYL

SEQ ID NO: 40 - protein, 12 aa, artificial sequence
  Synthetic construct
KPPHLAKLPFTT

SEQ ID NO: 41 - protein, 12 aa, artificial sequence
  Synthetic construct
NKRPPTSHRIHA

SEQ ID NO: 42 - protein, 12 aa, artificial sequence
  Synthetic construct
NLPRYQPPCKPL

SEQ ID NO: 43 - protein, 12 aa, artificial sequence
  Synthetic construct
RPPWKKPIPPSE

SEQ ID NO: 44 - protein, 12 aa, artificial sequence
  Synthetic construct
RQRPKDHFFSRP

SEQ ID NO: 45 - protein, 12 aa, artificial sequence
  Synthetic construct
SVPNKXVTVDGX

SEQ ID NO: 46 - protein, 12 aa, artificial sequence
  Synthetic construct
TTKWRHRAPVSP

SEQ ID NO: 47 - protein, 12 aa, artificial sequence
  Synthetic construct
WLGKNRIKPRAS

SEQ ID NO: 48 - protein, 12 aa, artificial sequence
  Synthetic construct
SNFKTPLPLTQS

SEQ ID NO: 49 - protein, 12 aa, artificial sequence
  Synthetic construct
SVSVGMKPSPRP

SEQ ID NO: 50 - protein, 7 aa, artificial sequence
  Synthetic construct
DLHTVYH

SEQ ID NO: 51 - protein, 7 aa, artificial sequence
  Synthetic construct
HIKPPTR

SEQ ID NO: 52 - protein, 7 aa, artificial sequence
  Synthetic construct
HPVWPAI

SEQ ID NO: 53 - protein, 7 aa, artificial sequence
  Synthetic construct
MPLYYLQ

SEQ ID NO: 54 - protein, 26 aa, artificial sequence
  Synthetic construct
HLTVPWRGGGSAVPFYSHSQITLPNH

SEQ ID NO: 55 - protein, 41 aa, artificial sequence
  Synthetic construct
GPHDTSSGGVRPNLHHTSKKEKRENRKVPFYSHSVTSRGNV

SEQ ID NO: 56 - protein, 7 aa, artificial sequence
  Synthetic construct
KHPTYRQ

SEQ ID NO: 57 - protein, 7 aa, artificial sequence
  Synthetic construct
HPMSAPR

SEQ ID NO: 58 - protein, 7 aa, artificial sequence
  Synthetic construct
MPKYYLQ

SEQ ID NO: 59 - protein, 7 aa, artificial sequence
  Synthetic construct
MHAHSIA

SEQ ID NO: 60 - protein, 12 aa, artificial sequence
  Synthetic construct
AKPISQHLQRGS

SEQ ID NO: 61 - protein, 12 aa, artificial sequence
  Synthetic construct
APPTPAAASATT

SEQ ID NO: 62 - protein, 12 aa, artificial sequence
  Synthetic construct
DPTEGARRTIMT

SEQ ID NO: 63 - protein, 12 aa, artificial sequence
  Synthetic construct
LDTSFPPVPFHA

SEQ ID NO: 64 - protein, 12 aa, artificial sequence
  Synthetic construct
LDTSFHQVPFHQ

SEQ ID NO: 65 - protein, 11 aa, artificial sequence
  Synthetic construct
LPRIANTWSPS

SEQ ID NO: 66 - protein, 12 aa, artificial sequence
  Synthetic construct
RTNAADHPAAVT

SEQ ID NO: 67 - protein, 12 aa, artificial sequence
  Synthetic construct
SLNWVTIPGPKI

SEQ ID NO: 68 - protein, 12 aa, artificial sequence
  Synthetic construct
TDMQAPTKSYSN

SEQ ID NO: 69 - protein, 12 aa, artificial sequence
  Synthetic construct
TIMTKSPSLSCG

SEQ ID NO: 70 - protein, 12 aa, artificial sequence
  Synthetic construct
TPALDGLRQPLR

SEQ ID NO: 71 - protein, 12 aa, artificial sequence
  Synthetic construct
TYPASRLPLLAP

SEQ ID NO: 72 - protein, 12 aa, artificial sequence
  Synthetic construct
AKTHKHPAPSYS

SEQ ID NO: 73 - protein, 12 aa, artificial sequence
  Synthetic construct
TDPTPFSISPER

SEQ ID NO: 74 - protein, 12 aa, artificial sequence
  Synthetic construct
SQNWQDSTSYSN

SEQ ID NO: 75 - protein, 12 aa, artificial sequence
  Synthetic construct
WHDKPQNSSKST

SEQ ID NO: 76 - protein, 12 aa, artificial sequence
  Synthetic construct
LDVESYKGTSMP

SEQ ID NO: 77 - protein, 7 aa, artificial sequence
  Synthetic construct
NTPKENW

SEQ ID NO: 78 - protein, 7 aa, artificial sequence
  Synthetic construct
NTPASNR

SEQ ID NO: 79 - protein, 7 aa, artificial sequence
  Synthetic construct
PRGMLST

SEQ ID NO: 80 - protein, 7 aa, artificial sequence
  Synthetic construct
PPTYLST

SEQ ID NO: 81 - protein, 12 aa, artificial sequence
  Synthetic construct
TIPTHRQHDYRS

SEQ ID NO: 82 - protein, 7 aa, artificial sequence
  Synthetic construct
TPPTHRL

SEQ ID NO: 83 - protein, 7 aa, artificial sequence
  Synthetic construct
LPTMSTP

SEQ ID NO: 84 - protein, 7 aa, artificial sequence
  Synthetic construct
LGTNSTP

SEQ ID NO: 85 - protein, 12 aa, artificial sequence
  Synthetic construct
TPLTGSTNLLSS

SEQ ID NO: 86 - protein, 7 aa, artificial sequence
  Synthetic construct
TPLTKET

SEQ ID NO: 87 - protein, 7 aa, artificial sequence
  Synthetic construct
KQSHNPP

SEQ ID NO: 88 - protein, 7 aa, artificial sequence
  Synthetic construct
QQSHNPP

SEQ ID NO: 89 - protein, 7 aa, artificial sequence
  Synthetic construct
TQPHNPP

SEQ ID NO: 90 - protein, 12 aa, artificial sequence
  Synthetic construct
STNLLRTSTVHP

SEQ ID NO: 91 - protein, 12 aa, artificial sequence
  Synthetic construct
HTQPSYSSTNLF

SEQ ID NO: 92 - protein, 7 aa, artificial sequence
  Synthetic construct
SLLSSHA

SEQ ID NO: 93 - protein, 12 aa, artificial sequence
  Synthetic construct
QQSSISLSSHAV

SEQ ID NO: 94 - protein, 7 aa, artificial sequence
  Synthetic construct
NASPSSL

SEQ ID NO: 95 - protein, 7 aa, artificial sequence
  Synthetic construct
HSPSSLR

SEQ ID NO: 96 - protein, 7 aa, artificial sequence
  Synthetic construct
KXSHHTH

SEQ ID NO: 97 - protein, 7 aa, artificial sequence
  Synthetic construct
EXSHHTH

SEQ ID NO: 98 - protein, 12 aa, artificial sequence
  Synthetic construct
SHHTHYGQPGPV

SEQ ID NO: 99 - protein, 7 aa, artificial sequence
  Synthetic construct
LESTSLL

SEQ ID NO: 100 - protein, 7 aa, artificial sequence
  Synthetic construct
DLTLPFH

SEQ ID NO: 101 - protein, 8 aa, artificial sequence
  Synthetic construct
RTNAADHP

SEQ ID NO: 102 - protein, 12 aa, artificial sequence
  Synthetic construct
IPWWNIRAPLNA

SEQ ID NO: 103 - protein, 18 aa, artificial sequence
  Synthetic construct
EQISGSLVAAPWEGEGER

SEQ ID NO: 104 - protein, 12 aa, artificial sequence
  synthetic hair-binding peptide
TPPELLHGAPRS

SEQ ID NO: 105 - protein, 18 aa, artificial sequence
  Synthetic construct
LDTSFHQVPFHQKRKRKD

SEQ ID NO: 106 - protein, 18 aa, artificial sequence
  Synthetic construct
EQISGSLVAAPWKRKRKD

SEQ ID NO: 107 - protein, 18 aa, artificial sequence
  Synthetic construct
TPPELLHGDPRSKRKRKD

SEQ ID NO: 108 - protein, 13 aa, artificial sequence
  Synthetic construct
NTSQLSTEGEGED

SEQ ID NO: 109 - protein, 13 aa, artificial sequence
  Synthetic construct
TPPELLHGDPRSC

SEQ ID NO: 110 - protein, 20 aa, artificial sequence
  synthetic hair-binding peptide
HINKTNPHQGNHHSEKTQRQ

SEQ ID NO: 111 - protein, 15 aa, artificial sequence
  Synthetic construct
HAHKNQKETHQRHAA

SEQ ID NO: 112 - protein, 15 aa, artificial sequence
  Synthetic construct
HEHKNQKETHQRHAA

SEQ ID NO: 113 - protein, 20 aa, artificial sequence
  Synthetic construct
HNHMQERYTEPQHSPSVNGL

SEQ ID NO: 114 - protein, 17 aa, artificial sequence
  Synthetic construct
THSTHNHGSPRHTNADA

SEQ ID NO: 115 - protein, 20 aa, artificial sequence
  synthetic hair-binding peptide
GSCVDTHKADSCVANNGPAT

SEQ ID NO: 116 - protein, 20 aa, artificial sequence
  synthetic hair-binding peptide
AQSQLPDKHSGLHERAPQRY

SEQ ID NO: 117 - protein, 20 aa, artificial sequence
  Synthetic construct
AQSQLPAKHSGLHERAPQRY

SEQ ID NO: 118 - protein, 20 aa, artificial sequence
  Synthetic construct
AQSQLPEKHSGLHERAPQRY

SEQ ID NO: 119 - protein, 20 aa, artificial sequence
  synthetic hair-binding peptide
TDMMHNHSDNSPPHRRSPRN

SEQ ID NO: 120 - protein, 20 aa, artificial sequence
  synthetic hair-binding peptide
TPPELAHTPHHLAQTRLTDR

SEQ ID NO: 121 - protein, 12 aa, artificial sequence
  Synthetic construct
RLLRLLRLLRLL

SEQ ID NO: 122 - protein, 12 aa, artificial sequence
  Synthetic construct
TPPELLHGEPRS

SEQ ID NO: 123 - protein, 12 aa, artificial sequence
  Synthetic construct
TPPELLHGAPRS

SEQ ID NO: 124 - protein, 12 aa, artificial sequence
  Synthetic construct
EQISGSLVAAPW

SEQ ID NO: 125 - protein, 12 aa, artificial sequence
  Synthetic construct
NEVPARNAPWLV

SEQ ID NO: 126 - protein, 13 aa, artificial sequence
  Synthetic construct
NSPGYQADSVAIG

SEQ ID NO: 127 - protein, 12 aa, artificial sequence
  Synthetic construct
AKPISQHLQRGS

SEQ ID NO: 128 - protein, 12 aa, artificial sequence
  Synthetic construct
LDTSFPPVPFHA

SEQ ID NO: 129 - protein, 12 aa, artificial sequence
  Synthetic construct
SLNWVTIPGPKI

SEQ ID NO: 130 - protein, 12 aa, artificial sequence
  Synthetic construct
TQDSAQKSPSPL

SEQ ID NO: 131 - protein, 12 aa, artificial sequence
  Synthetic construct
KELQTRNVVQRE

SEQ ID NO: 132 - protein, 12 aa, artificial sequence
  Synthetic construct
QRNSPPAMSRRD

SEQ ID NO: 133 - protein, 12 aa, artificial sequence
  Synthetic construct
TPTANQFTQSVP

SEQ ID NO: 134 - protein, 12 aa, artificial sequence
  Synthetic construct
AAGLSQKHERNR

SEQ ID NO: 135 - protein, 12 aa, artificial sequence
  Synthetic construct
ETVHQTPLSDRP

SEQ ID NO: 136 - protein, 12 aa, artificial sequence
  Synthetic construct
KNFPQQKEFPLS

SEQ ID NO: 137 - protein, 12 aa, artificial sequence
  Synthetic construct
LPALHIQRHPRM

SEQ ID NO: 138 - protein, 12 aa, artificial sequence
  Synthetic construct
QPSHSQSHNLRS

SEQ ID NO: 139 - protein, 12 aa, artificial sequence
  Synthetic construct
RGSQKSKPPRPP

SEQ ID NO: 140 - protein, 12 aa, artificial sequence
  Synthetic construct
THTQKTPLLYYH

SEQ ID NO: 141 - protein, 12 aa, artificial sequence
  Synthetic construct
TKGSSQAILKST

SEQ ID NO: 142 - protein, 7 aa, artificial sequence
  Synthetic construct
TAATTSP

SEQ ID NO: 143 - protein, 7 aa, artificial sequence
  Synthetic construct
LGIPQNL

SEQ ID NO: 144 - protein, 20 aa, artificial sequence
  Synthetic construct
THSTHNHGSPRHTNADAGNP

SEQ ID NO: 145 - protein, 20 aa, artificial sequence
  Synthetic construct
QQHKVHHQNPDRSTQDAHHS

SEQ ID NO: 146 - protein, 15 aa, artificial sequence
  Synthetic construct
HHGTHHNATKQKNHV

SEQ ID NO: 147 - protein, 15 aa, artificial sequence
  Synthetic construct
STLHKYKSQDPTPHH

SEQ ID NO: 148 - protein, 12 aa, artificial sequence
  Synthetic construct
SVSVGMKPSPRP

SEQ ID NO: 149 - protein, 12 aa, artificial sequence
  synthetic hair-binding peptide
TPPTNVLMLATK

SEQ ID NO: 150 - protein, 12 aa, artificial sequence
  Synthetic construct
TPPELLHGDPRS

SEQ ID NO: 151 - protein, 7 aa, artificial sequence
  synthetic hair-binding peptide
NTSQLST

SEQ ID NO: 152 - protein, 15 aa, artificial sequence
  Synthetic construct
STLHKYKSQDPTPHH

SEQ ID NO: 153 - protein, 12 aa, artificial sequence
  synthetic hair-binding peptide
GMPAMHWIHPFA

SEQ ID NO: 154 - protein, 15 aa, artificial sequence
  synthetic hair-binding peptide
HDHKNQKETHQRHAA

SEQ ID NO: 155 - protein, 20 aa, artificial sequence
  Synthetic construct
HNHMQERYTDPQHSPSVNGL

SEQ ID NO: 156 - protein, 20 aa, artificial sequence
  synthetic hair-binding peptide
TAEIQSSKNPNPHPQRSWTN

SEQ ID NO: 157 - protein, 12 aa, artificial sequence
  Synthetic construct
KRGRHKRPKRHK

SEQ ID NO: 158 - protein, 7 aa, artificial sequence
  Synthetic construct
RLLRLLR

SEQ ID NO: 159 - protein, 12 aa, artificial sequence
  Synthetic construct
HKPRGGRKKALH

SEQ ID NO: 160 - protein, 18 aa, artificial sequence
  Synthetic construct
KPRPPHGKKHRPKHRPKK

SEQ ID NO: 161 - protein, 18 aa, artificial sequence
  Synthetic construct
RGRPKKGHGKRPGHRARK

SEQ ID NO: 162 - protein, 12 aa, artificial sequence
  Synthetic construct
TPFHSPENAPGS

SEQ ID NO: 163 - protein, 13 aa, artificial sequence
  Synthetic construct
TPFHSPENAPGSK

SEQ ID NO: 164 - protein, 16 aa, artificial sequence
  Synthetic construct
TPFHSPENAPGSGGGS

SEQ ID NO: 165 - protein, 17 aa, artificial sequence
  Synthetic construct
TPFHSPENAPGSGGGSS

SEQ ID NO: 166 - protein, 15 aa, artificial sequence
  Synthetic construct
TPFHSPENAPGSGGG

SEQ ID NO: 167 - protein, 7 aa, artificial sequence
  Synthetic construct
FTQSLPR

SEQ ID NO: 168 - protein, 12 aa, artificial sequence
  Synthetic construct
KQATFPPNPTAY

SEQ ID NO: 169 - protein, 12 aa, artificial sequence
  Synthetic construct
HGHMVSTSQLSI

SEQ ID NO: 170 - protein, 7 aa, artificial sequence
  Synthetic construct
LSPSRMK

SEQ ID NO: 171 - protein, 7 aa, artificial sequence
  Synthetic construct
LPIPRMK

SEQ ID NO: 172 - protein, 7 aa, artificial sequence
  Synthetic construct
HQRPYLT

SEQ ID NO: 173 - protein, 7 aa, artificial sequence
  Synthetic construct
FPPLLRL

SEQ ID NO: 174 - protein, 7 aa, artificial sequence
  Synthetic construct
QATFMYN

SEQ ID NO: 175 - protein, 11 aa, artificial sequence
  Synthetic construct
VLTSQLPNHSM

SEQ ID NO: 176 - protein, 7 aa, artificial sequence
  Synthetic construct
HSTAYLT

SEQ ID NO: 177 - protein, 12 aa, artificial sequence
  Synthetic construct
APQQRPMKTFNT

SEQ ID NO: 178 - protein, 12 aa, artificial sequence
  Synthetic construct
APQQRPMKTVQY

SEQ ID NO: 179 - protein, 7 aa, artificial sequence
  Synthetic construct
PPWLDLL

SEQ ID NO: 180 - protein, 7 aa, artificial sequence
  Synthetic construct
PPWTFPL

SEQ ID NO: 181 - protein, 7 aa, artificial sequence
  Synthetic construct
SVTHLTS

SEQ ID NO: 182 - protein, 7 aa, artificial sequence
  Synthetic construct
VITRLTS

SEQ ID NO: 183 - protein, 12 aa, artificial sequence
  Synthetic construct
DLKPPLLALSKV

SEQ ID NO: 184 - protein, 12 aa, artificial sequence
  Synthetic construct
SHPSGALQEGTF

SEQ ID NO: 185 - protein, 12 aa, artificial sequence
  Synthetic construct
FPLTSKPSGACT

SEQ ID NO: 186 - protein, 12 aa, artificial sequence
  Synthetic construct
DLKPPLLALSKV

SEQ ID NO: 187 - protein, 7 aa, artificial sequence
  Synthetic construct
PLLALHS

SEQ ID NO: 188 - protein, 7 aa, artificial sequence
  Synthetic construct
VPISTQI

SEQ ID NO: 189 - protein, 12 aa, artificial sequence
  Synthetic construct
YAKQHYPISTFK

SEQ ID NO: 190 - protein, 7 aa, artificial sequence
  Synthetic construct
HSTAYLT

SEQ ID NO: 191 - protein, 12 aa, artificial sequence
  Synthetic construct
STAYLVAMSAAP

SEQ ID NO: 192 - protein, 12 aa, artificial sequence
  Synthetic construct
SVSVGMKPSPRP

SEQ ID NO: 193 - protein, 12 aa, artificial sequence
  Synthetic construct
TMGFTAPRFPHY

SEQ ID NO: 194 - protein, 12 aa, artificial sequence
  Synthetic construct
NLQHSVGTSPVW

SEQ ID NO: 195 - protein, 15 aa, artificial sequence
  Synthetic construct
QLSYHAYPQANHHAP

SEQ ID NO: 196 - protein, 12 aa, artificial sequence
  Synthetic construct
NQAASITKRVPY

SEQ ID NO: 197 - protein, 14 aa, artificial sequence
  Synthetic construct
SGCHLVYDNGFCDH

SEQ ID NO: 198 - protein, 14 aa, artificial sequence
  Synthetic construct
ASCPSASHADPCAH

SEQ ID NO: 199 - protein, 14 aa, artificial sequence
  Synthetic construct
NLCDSARDSPRCKV

SEQ ID NO: 200 - protein, 12 aa, artificial sequence
  Synthetic construct
NHSNWKTAADFL

SEQ ID NO: 201 - protein, 12 aa, artificial sequence
  Synthetic construct
GSSTVGRPLSYE

SEQ ID NO: 202 - protein, 12 aa, artificial sequence
  Synthetic construct
SDTISRLHVSMT

SEQ ID NO: 203 - protein, 12 aa, artificial sequence
  Synthetic construct
SPLTVPYERKLL

SEQ ID NO: 204 - protein, 12 aa, artificial sequence
  Synthetic construct
SPYPSWSTPAGR

SEQ ID NO: 205 - protein, 12 aa, artificial sequence
  Synthetic construct
VQPITNTRYEGG

SEQ ID NO: 206 - protein, 12 aa, artificial sequence
  Synthetic construct
WPMHPEKGSRWS

SEQ ID NO: 207 - protein, 14 aa, artificial sequence
  Synthetic construct
DACSGNGHPNNCDR

SEQ ID NO: 208 - protein, 14 aa, artificial sequence
  Synthetic construct
DHCLGRQLQPVCYP

SEQ ID NO: 209 - protein, 14 aa, artificial sequence
  Synthetic construct
DWCDTIIPGRTCHG

SEQ ID NO: 210 - protein, 12 aa, artificial sequence
  Synthetic construct
ALPRIANTWSPS

SEQ ID NO: 211 - protein, 12 aa, artificial sequence
  Synthetic construct
YPSFSPTYRPAF

SEQ ID NO: 212 - protein, 20 aa, artificial sequence
  Synthetic construct
AHPESLGIKYALDGNSDPHA

SEQ ID NO: 213 - protein, 20 aa, artificial sequence
  Synthetic construct
ASVSNYPPIHHLATSNTTVN

SEQ ID NO: 214 - protein, 14 aa, artificial sequence
  Synthetic construct
DECMEPLNAAHCWR

SEQ ID NO: 215 - protein, 14 aa, artificial sequence
  Synthetic construct
DECMHGSDVEFCTS

SEQ ID NO: 216 - protein, 14 aa, artificial sequence
  Synthetic construct
DLCSMQMMNTGCHY

SEQ ID NO: 217 - protein, 14 aa, artificial sequence
  Synthetic construct
DLCSSPSTWGSCIR

SEQ ID NO: 218 - protein, 20 aa, artificial sequence
  Synthetic construct
DPNESNYENATTVSQPTRHL

SEQ ID NO: 219 - protein, 20 aa, artificial sequence
  Synthetic construct
EPTHPTMRAQMHQSLRSSSP

SEQ ID NO: 220 - protein, 20 aa, artificial sequence
  Synthetic construct
GNTDTTPPNAVMEPTVQHKW

SEQ ID NO: 221 - protein, 15 aa, artificial sequence
  Synthetic construct
NGPDMVQSVGKHKNS

SEQ ID NO: 222 - protein, 15 aa, artificial sequence
  Synthetic construct
NGPEVRQIPANFEKL

SEQ ID NO: 223 - protein, 20 aa, artificial sequence
  Synthetic construct
NNTSADNPPETDSKHHLSMS

SEQ ID NO: 224 - protein, 20 aa, artificial sequence
  Synthetic construct
NNTWPEGAGHTMPSTNIRQA

SEQ ID NO: 225 - protein, 20 aa, artificial sequence
  Synthetic construct
NPTATPHMKDPMHSNAHSSA

SEQ ID NO: 226 - protein, 20 aa, artificial sequence
  Synthetic construct
NPTDHIPANSTNSRVSKGNT

SEQ ID NO: 227 - protein, 15 aa, artificial sequence
  Synthetic construct
NPTDSTHMMHARNHE

SEQ ID NO: 228 - protein, 14 aa, artificial sequence
  Synthetic construct
QHCITERLHPPCTK

SEQ ID NO: 229 - protein, 14 aa, artificial sequence
  Synthetic construct
TPCAPASFNPHCSR

SEQ ID NO: 230 - protein, 14 aa, artificial sequence
  Synthetic construct
TPCATYPHFSGCRA

SEQ ID NO: 231 - protein, 20 aa, artificial sequence
  Synthetic construct
WCTDFCTRSTPTSTSRSTTS

SEQ ID NO: 232 - protein, 20 aa, artificial sequence
  Synthetic construct
APPLKTYMQERELTMSQNKD

SEQ ID NO: 233 - protein, 20 aa, artificial sequence
  Synthetic construct
EPPTRTRVNNHTVTVQAQQH

SEQ ID NO: 234 - protein, 14 aa, artificial sequence
  Synthetic construct
GYCLRGDEPAVCSG

SEQ ID NO: 235 - protein, 20 aa, artificial sequence
  Synthetic construct
LSSKDFGVTNTDQRTYDYTT

SEQ ID NO: 236 - protein, 14 aa, artificial sequence
  Synthetic construct
NFCETQLDLSVCTV

SEQ ID NO: 237 - protein, 14 aa, artificial sequence
  Synthetic construct
NTCQPTKNATPCSA

SEQ ID NO: 238 - protein, 20 aa, artificial sequence
  Synthetic construct
PSEPERRDRNIAANAGRFNT

SEQ ID NO: 239 - protein, 18 aa, artificial sequence
  Synthetic construct
THNMSHFPPSGHPKRTAT

SEQ ID NO: 240 - protein, 14 aa, artificial sequence
  Synthetic construct
TTCPTMGTYHVCWL

SEQ ID NO: 241 - protein, 20 aa, artificial sequence
  Synthetic construct
YCADHTPDPANPNKICGYSH

SEQ ID NO: 242 - protein, 20 aa, artificial sequence
  Synthetic construct
AANPHTEWDRDAFQLAMPPK

SEQ ID NO: 243 - protein, 20 aa, artificial sequence
  Synthetic construct
DLHPMDPSNKRPDNPSDLHT

SEQ ID NO: 244 - protein, 14 aa, artificial sequence
  Synthetic construct
ESCVSNALMNQCIY

SEQ ID NO: 245 - protein, 20 aa, artificial sequence
  Synthetic construct
HNKADSWDPDLPPHAGMSLG

SEQ ID NO: 246 - protein, 20 aa, artificial sequence
  Synthetic construct
LNDQRKPGPPTMPTHSPAVG

SEQ ID NO: 247 - protein, 14 aa, artificial sequence
  Synthetic construct
NTCATSPNSYTCSN

SEQ ID NO: 248 - protein, 14 aa, artificial sequence
  Synthetic construct
SDCTAGLVPPLCAT

SEQ ID NO: 249 - protein, 20 aa, artificial sequence
  Synthetic construct
TIESSQHSRTHQQNYGSTKT

SEQ ID NO: 250 - protein, 20 aa, artificial sequence
  Synthetic construct
VGTMKQHPTTTQPPRVSATN

SEQ ID NO: 251 - protein, 20 aa, artificial sequence
  Synthetic construct
YSETPNDQKPNPHYKVSGTK

SEQ ID NO: 252 - protein, 8 aa, Artificial Sequence
  Caspase 3 cleavage site
LESGDEVD

SEQ ID NO: 253 - protein, 7 aa, artificial sequence
  peptide linker
GGKGGAG

SEQ ID NO: 254 - protein, 7 aa, artificial sequence
  peptide linker
GGAGGAG

SEQ ID NO: 255 - protein, 33 aa, artificial sequence
  peptide linker
EPEPEPEPIPEPPKEAPVVIEKPKPKPKPKPKP

SEQ ID NO: 256 - protein, 21 aa, artificial sequence
  peptide linker
GKGKGKGKGKGKGKGKGKGKG

SEQ ID NO: 257 - protein, 20 aa, artificial sequence
  peptide linker
KGGGSKGGGSKGGGSKGGGS

SEQ ID NO: 258 - protein, 7 aa, artificial sequence
  peptide bridge
GGAGGAV

SEQ ID NO: 259 - protein, 8 aa, artificial sequence
  peptide bridge
GSGGGGSP

SEQ ID NO: 260 - protein, 16 aa, artificial sequence
  peptide bridge
GSGGGGSPGSGGGGSP

SEQ ID NO: 261 - protein, 8 aa, artificial sequence
  peptide bridge
GSGGGGSP

SEQ ID NO: 262 - DNA, 20 bp, artificial sequence
  primer
ccctcatagttagcgtaacg

SEQ ID NO: 263 - protein, 20 aa, Artificial Sequence
  Synthetic oral-surface binding peptide
NGNNHTDIPNRSSYTGGSFA

SEQ ID NO: 264 - protein, 20 aa, Artificial Sequence
  Synthetic oral-surface binding peptide
TMTNHVYNSYTEKHSSTHRS

SEQ ID NO: 265 - protein, 20 aa, Artificial Sequence
  Synthetic oral-surface binding peptide
TTYHYKNIYQESYQQRNPAV

SEQ ID NO: 266 - protein, 20 aa, Artificial Sequence
  Synthetic oral-surface binding peptide
VEPATKNMREARSSTQMRRI

SEQ ID NO: 267 - protein, 20 aa, Artificial Sequence
  Synthetic oral-surface binding peptide
YLLPKDQTTAPQVTPIVQHK

SEQ ID NO: 268 - protein, 18 aa, Artificial Sequence
  Synthetic oral-surface binding peptide
ASNLDSTFTAINTPACCT

SEQ ID NO: 269 - protein, 18 aa, Artificial Sequence
  Synthetic oral-surface binding peptide
EFPYYNDNPPNPERHTLR

SEQ ID NO: 270 - protein, 18 aa, Artificial Sequence
  Synthetic oral-surface binding peptide
GMPTRYYHNTPPHLTPKF

SEQ ID NO: 271 - protein, 18 aa, Artificial Sequence
  Synthetic oral-surface binding peptide
HKNAIQPVNDATTLDTTM

SEQ ID NO: 272 - protein, 15 aa, Artificial Sequence
  Synthetic oral-surface binding peptide
AVVPADLNDHANHLS

SEQ ID NO: 273 - protein, 15 aa, Artificial Sequence
  Synthetic oral-surface binding peptide
DLGTFPNRTLKMAAH

SEQ ID NO: 274 - protein, 15 aa, Artificial Sequence
  Synthetic oral-surface binding peptide
FDGIGLGTATRHQNR

SEQ ID NO: 275 - protein, 15 aa, Artificial Sequence
  Synthetic oral-surface binding peptide
QAAQVHMMQHSRPTT

SEQ ID NO: 276 - protein, 18 aa, Artificial Sequence
  Synthetic oral-surface binding peptide
SEARARTFNDHTTPMPII

SEQ ID NO: 277 - protein, 18 aa, Artificial Sequence
  Synthetic oral-surface binding peptide
ELDHDSRHYMNGLQRKVT

SEQ ID NO: 278 - protein, 18 aa, Artificial Sequence
  Synthetic oral-surface binding peptide
GPQHVLMQDTHQGYAFDN

SEQ ID NO: 279 - protein, 20 aa, Artificial Sequence
  Synthetic oral-surface binding peptide
TTGSSSQADTSASMSIVPAH

SEQ ID NO: 280 - protein, 18 aa, Artificial Sequence
  Synthetic oral-surface binding peptide
KAPIANMLQPHSYQYSVA

SEQ ID NO: 281 - protein, 18 aa, Artificial Sequence
  Synthetic oral-surface binding peptide
TYQGVPSWPAVIDDAIRR

SEQ ID NO: 282 - protein, 18 aa, Artificial Sequence
  Synthetic oral-surface binding peptide
VNPNWVETQALHQPPGNT

SEQ ID NO: 283 - protein, 20 aa, Artificial Sequence
  Synthetic oral-surface binding peptide
DHNNRQHAVEVRENKTHTAR

SEQ ID NO: 284 - protein, 18 aa, Artificial Sequence
  Synthetic oral-surface binding peptide
IYPNESMSTSNVRGPYHP

SEQ ID NO: 285 - protein, 20 aa, Artificial Sequence
  Synthetic oral-surface binding peptide
HDPNHLTHQARTIYRNANHT

SEQ ID NO: 286 - protein, 15 aa, Artificial Sequence
  Synthetic oral-surface binding peptide
SNATMYNIQSHSHHQ

SEQ ID NO: 287 - protein, 15 aa, Artificial Sequence
  Synthetic oral-surface binding peptide
ANELSTYAQTNPGSG

SEQ ID NO: 288 - protein, 15 aa, Artificial Sequence
  Synthetic oral-surface binding peptide
DTIHPNKMKSPSSPL

SEQ ID NO: 289 - protein, 20 aa, Artificial Sequence
  Synthetic oral-surface binding peptide
APPTYQTASYPHNLPSKRKM

SEQ ID NO: 290 - protein, 20 aa, Artificial Sequence
  Synthetic oral-surface binding peptide
QVPDYLSPTHQKKAFLEIPT

SEQ ID NO: 291 - protein, 20 aa, Artificial Sequence
  Synthetic oral-surface binding peptide
TNDLHANPFTGTYIAPDPTS

SEQ ID NO: 292 - protein, 20 aa, Artificial Sequence
  Synthetic oral-surface binding peptide
HKNENIMQYNVNDRWHITPA

SEQ ID NO: 293 - protein, 18 aa, Artificial Sequence
  Synthetic oral-surface binding peptide
IDGPHHSPVHRYHTPSIT

SEQ ID NO: 294 - protein, 15 aa, Artificial Sequence
  Synthetic peptide
IPWWNIRAPLNAGGG

SEQ ID NO: 295 - protein, 32 aa, artificial sequence
  synthetic construct
PSSSRPTTYHYKNIYQESYQQRNPAVHHHHHH

SEQ ID NO: 296 - protein, 71 aa, artificial sequence
  synthetic construct
PSSSRPTTYHYKNIYQESYQQRNPAVGPEPEPEPEPIPEPPKEAPVVIEKPKPKPKPKPKPPAGPHHHHHH

SEQ ID NO: 297 - protein, 95 aa, artificial sequence
  synthetic construct
PSSSRPTTYHYKNIYQESYQQRNPAVGPEPEPEPEPIPEPPKEAPVVIEKPKPKPKPKPKPPASSRPTTYHYKNIYQESYQQRNPAVGPHHHHHH

SEQ ID NO: 298 - protein, 135 aa, artificial sequence
  synthetic construct
PSSRPTTYHYKNIYQESYQQRNPAVGPEPEPEPEPIPEPPKEAPVVIEKPKPKPKPKPKPPASSRPTTYHYKNIYQESYQQRNPAVGSSGPGSPYKHERHYSQPLKVRHGSGYKHERHYSQPLKVRHGPHHHHHH